SEQUENCE LISTING (2 sequences)

SEQ ID NO: 1 - DNA, 27 bp, sv40 virus
taggcttctagaaaaagctttgcaaag

SEQ ID NO: 2 - DNA, 28 bp, sv40 virus
acaattgggcccattttatgtttcaggt

(19) United States
(12) Patent Application Publication
(10) Pub. No.: US 2005/0118699 A1
(43) Pub. Date: Jun. 2, 2005

(54) Packaging complementation cell-line for sv-40 vectors

(76) Inventors: Ariella Oppenheim, Jerusalem (IL); Uri Arad, Jerusalem (IL)

Correspondence Address: Cooper & Dunham, 1185 Avenue of the Americas, New York, NY, 10036, US

(21) Appl. No.: 10/489,989

(22) PCT Filed: Sep. 12, 2002
(86) PCT No.: PCT/IL02/00757

(30) Foreign Application Priority Data
Sep. 16, 2001 (IL) 145463

(51) Int. Cl. C12N 7/00; C12N 5/08; C12N 15/867

(52) U.S. Cl. 435/235.1; 435/456; 435/372

(57) ABSTRACT

The present invention relates to a complementation SV40 packaging cell line for in-trans complementation of viral T-antigen (T-Ag). This cell line eliminates the generation of viable virus carrying the T-Ag gene, by reducing homologous recombination in packaging process of an SV40 vector, wherein said cell line is transformed with at least one expression cassette having minimal sequence identity to SV40 sequences comprised within said SV40 viral vector. The expression cassette of the invention comprises nucleic acid encoding SV40 T-Ag under the control of a heterologous promoter, heterologous termination signal; and optionally additional operably linked control elements and/or selectable markers. The invention further relates to methods for the preparation of packaging cell line, processes for production of a safe preparation of SV40 viral vectors and compositions comprising the same.

### FIELD OF THE INVENTION

The present invention relates to an SV40 wild-type-free packaging cell line for SV40 vectors for in-trans complementation of SV40 T-antigen (T-Ag). More specifically, this cell line was engineered to carry minimal sequence identity to SV40 sequences comprised within an SV40 viral vector, and it therefore eliminates homologous recombination of the vector with SV40 DNA that is integrated within said cell. The complementation cells are used for the production of SV40 viral vectors having no wild type recombinants contamination. The invention further provides process for the production of such safe SV40 viral vectors and compositions thereof

### BACKGROUND OF THE INVENTION

All publications mentioned throughout this application are fully incorporated herein by reference, including all references cited therein.

SV40 is a promising viral vector for efficient gene transfer into a variety of human tissues including the bone marrow [Oppenheim, A., et al., Proc. Nat. Acad. of Sci. USA 83, 6925-9 (1986); Rund, D., et al., Human Gene Therapy 9, 649-57 (1998)] and the liver [Strayer, D. S. and Zern, M. A. Seminars in Liver Disease 19, 71-81 (1999)], both critical target organs for the treatment of many diseases. Obstacles such as the inability to transduce non-dividing cells, immunogenicity or transient gene expression hinder the effectiveness of currently available viral gene transfer vectors. SV40 vectors infect non-dividing as well as cycling cells and appear to be non-immunogenic [Strayer, ibid (1999)], thus making them important candidates for therapeutic gene transfer.

In SV40 T-antigen replacement vectors, the viral early region (the T-antigens) is replaced by a transgene. The large SV40 T-antigen activates both viral DNA replication and transcription of the viral late genes. Hence, deletion of T-antigen ensures the production of replication incompetent vectors that are also incapable of late gene expression. Thus, the absence of T-antigen sequences in SV40 vectors also eliminates the inherent immunogenicity of both the T-antigen protein and the structural late proteins.

Deletion of the T-(tumor) antigen also eliminates the potential risk of oncogenicity. This is because the ability of SV40 to transform established cell lines, to immortalize primary cell cultures and to induce tumors in immune-compromised rodents, are functions of the large T-antigen. The T-antigen of SV40 disrupts cell growth control mechanisms by binding and inactivating the tumor suppressor proteins p53 and the pRb family members.

Hundreds of millions of people were inadvertently exposed to wild-type SV40 in contaminated poliovirus vaccines. After more than 30 years of follow-up, epidemiological studies have found no evidence to suggest that SV40 is associated with human malignancy [Strickler, H. D. et al., J. Am. Med. Assoc. 279, 292-5 (1998)]. Still, the reports of some cases of human tumors with SV40 T-antigen sequences have led to concern regarding the possible etiological role of SV40 T-antigen in certain human cancers [reviewed in Butel, J. S. and Lednicky, J. A. Journal of the National Cancer Institute 91, 119-34 (1999)]. Hence, the exclusion of contaminating T-antigen sequences is a major safety issue in the development of SV40-based vectors.

T-antigen is a major immunogenic component of SV40, eliciting cellular and humoral immune responses targeted towards infected cells. Viral late gene expression depends upon both T-antigen-mediated DNA replication and upon T-antigen-mediated transactivation. Therefore, lack of T-antigen sequences in SV40 vectors is necessary to eliminate both the inherent immunogenicity of the T-antigen protein and the immunogenicity of the structural late proteins. T-antigen replacement vectors can be propagated in cell-lines that are permissive for lytic growth of SV40, and supply the T-antigen in trans. The most commonly used cell line is COS, constructed by Gluzman [Gluzman, Y. Cell 23, 175-82 (1981)]. COS cells were derived by transformation of CV-1 simian cells with a mutant of SV40 incapable of DNA replication due to a 6 nucleotide deletion at the viral origin of replication (ori). COS-1 cells contain one copy of the T-antigen structural gene and flanking sequences [Gluzman, ibid. (1981)], whereas COS-7 cells contain approximately 5-7 copies. In addition to more copies, COS-7 cells also have a longer fragment of integrated SV40 [Jasin, M., et al. Cell 43, 695-703 (1985)]. Another established cell line that expresses the T-antigen is CMT4 [Gerard, R. D. and Gluzman, Y. Molecular and Cellular Biology 5, 3231-40 (1985)] which was designed to express high, inducible levels of T-antigen under control of the mouse metallothionein promoter. The CMT cell lines were also prepared with DNA containing the entire SV40 genome.

In the original paper describing the COS cell-lines [Gluzman, ibid. (1981)], Gluzman reported that passaging of T-antigen mutants in COS cells resulted in the appearance of recombinant wild-type virus, at a low frequency. The emergence of such recombinants, usually detected as plaque forming units (PFU) on CV-1 cells (that do not contain T-antigen), was confirmed by a number of investigators [Shaul, Y., et al., Proc. Nat. Acad. Sci. USA., 82, 3781-4 (1985); Jasin, ibid. (1985); Oppenheim, A. and Peleg, A. Gene 77, 79-86 (1989)]. The generation of recombinants, though erratic, may reach a ratio of more than 1:1000 PFU to T-antigen deleted vectors in three passages in COS-1 cells [Oppenheim, ibid. (1989)]. It was observed that recombination in COS-7 is much higher than in COS-1 [Jasin, ibid. (1985)]. Detailed analysis [Shaul, ibid. (1985)] has shown that reacquisition of T-antigen gene most probably occurs by two homologous recombination events at sites of sequence identity encompassing the region of the T-antigen gene. Therefore, utilization of SV40 for medical purpose is prohibited due to the presence of viable SV40 virus.

The present invention describes the construction of cell lines that allow the production of SV40 viruses and pseudoviruses that are safe for medical use.

It is to be appreciated that notwithstanding the great interest in SV40 viral vectors for research and gene therapy, the present invention discloses for the first time creation of such complementation cell line. It should be further emphasized that creation of such cell line was not trivial. The problem of wild type SV40 recombinants was realized following the construction of the first set of complementation cell lines, COS, in 1981. The improved complementation cell line, CMT4, was constructed in 1985, but did not solve this problem. In spite of the interest in SV40 vectors for gene therapy, and the realization of the severe health risk imposed by presence of viable SV40, no progress has been made in the field for over 15 years. Furthermore, in the present study the inventors have confirmed that propagation of SV40 vectors in COS-1 and CMT4 cells resulted in the emergence of replication competent SV40.

The creation of the present complementation cell line involved the inventiveness of creating the expression cassette of the invention, which comprises only minimal sequence identity to SV40 sequences. This was not trivial because of the overlap of T-antigen coding sequence with the critical regulatory elements present in the vector which is absolutely required for its function. Construction of cell lines comprising this novel and inventive expression cassette eliminate formation of viable recombinant SV40 viruses and thus allow production of safe SV40 vectors which are particularly suitable for medical use.

In order to eliminate the generation of viable SV40, packaging cell-lines that can express the T-Antigen but contain minimal sequence identity with the SV40 vector have been constructed. These packaging cell-lines indeed showed that the generation of T-antigen positive revertants was eliminated. One of the cell-lines, COT18, allowed significant propagation of high titer stocks with minimal loss of transducing activity.

It is therefore an object of the invention to provide safe complementation SV40 packaging cell lines that can be used for packaging SV40 viral vectors without generating recombinant viable viruses that carry and can express the T-antigen.

### SUMMARY OF THE INVENTION

In a first aspect, the present invention relates to a complementation SV40 packaging cell line for in-trans complementation of viral T-antigen (T-Ag). This cell line is stably transformed with at least one expression cassette having minimal sequence identity to SV40 sequences comprised within an SV40 viral vector. The sequence identity is lower then the required for homologous recombination in said cells. This strategy eliminates the production of viable SV40 viruses during the preparation of SV40 viral or SV40 pseudoviral vector stocks. Only the T-antigen coding sequence, which is contained within the expression cassette, is introduced into the complementation cells of the invention. The expression cassette comprises a nucleic acid sequence coding for SV40 T-Ag, a heterologous promoter optionally the promoter is an inducible one, a heterologous termination signal, preferably polyadenylation sequence and optionally additional operably linked control elements and/or selectable markers.

In one embodiment, the packaging cell line of the invention is optionally further transformed with an additional expression cassette comprising a gene coding for a selectable marker.

In a particularly preferred embodiment, in addition to the T-Ag encoding sequence, the complementation cells of the invention have minimal sequence identity to the SV40 viral vectors. According to the invention, the sequence identity to the SV40 viral vectors which is below the identity required for efficient homologous recombination of the SV40 sequences comprised within an SV40 viral vector and sequences of the integrated virus in said cell line. According to a preferred embodiment, the SV40 sequences comprised within the cells and the SV40 sequences comprised within said SV40 viral vector share a sequence identity of between 0 and about 300 nucleotides. Preferably, between 0 to 250 nucleotides, more preferably between 0 to 100 nucleotides. Most preferably, between 0 to 10 and specifically most preferably the complementation cells of the invention do not contain any sequence identical with the SV40 sequence comprised within the SV40 vectors.

In a particular specific embodiment, the SV40 sequences comprised within the cells and the SV40 viral vector share 83 nucleotides corresponding to nucleotide 2770 to 2687 of the SV40 genome as denoted by GenBank Accession Number J20400.

In yet another specifically preferred embodiment, the T-Ag is expressed in the packaging cell line of the invention, under the transcriptional control of a heterologous promoter, preferably an inducible promoter. More preferably, this inducible promoter is the mouse inducible metalloprotein promoter. In a further preferred embodiment, the T-Ag expressed in the packaging cell line of the invention is placed under the control of a heterologous termination signal. More particularly, the heterologous termination signal may be a bovine growth hormone polyadenylation signal.

In a particularly preferred specific embodiment, the SV40 T-Ag region expressed by the packaging cell line of the invention, is a nucleic acid fragment corresponding to nucleotides 5179 to 2687 (nucleotides of the coding sequence itself are 5163-2694) of the SV40 genome as denoted by GenBank Accession Number J20400.

In yet another specifically preferred embodiment the expression cassette is comprised within a plasmid. A preferred plasmid is the plasmid designated pUMTB. Thus, according to a specific embodiment, the packaging cell line of the invention is transformed with the plasmid pUMTB. This plasmid comprises an expression cassette comprising an SV40 T-Ag encoding fragment corresponding to nucleotides 5179 to 2687 of the SV40 genome and expressed under the control of the mouse inducible metalloprotein promoter and a bovine growth hormone polyadenylation signal.

According to another specific embodiment, the packaging cell line of the invention is further transformed with an additional expression cassette comprising a gene coding for a selectable marker. This cassette according to a specific embodiment is comprised within a plasmid, preferably the plasmid designated pLN, and comprises a sequence coding for neomycine resistance sequence without any sequences that are homologous to SV40.

Thus, in a specifically preferred embodiment, the packaging cell line of the invention is transformed with the plasmids pUMTB and pLN.

The packaging cell line of the invention may be derived from any one of primate SV40 permissive or semi-permissive primary cells and cell lines. According to more specific embodiment, the complementation cell line of the invention may derive from a cell line selected from the group consisting of Vero, CV-1, Hela and BSC1.

In a particularly preferred embodiment, the packaging cell line of the invention is derived from CV-1 cell line. Of particular interest are the CV-1 derived packaging cell lines selected from the group consisting of COT2, COT4 and COT18. Most preferred CV-1 derived cell line is the COT18. This cell line was deposited under DSM Accession No. ACC2525. It is to be mentioned that this complementation cell line and any cell lines derived therefrom are within the scope of the present invention.

In a second aspect, the present invention relates to an expression cassette for the production of a complementation SV40 packaging cell line. This expression cassette has minimal sequence identity to SV40 sequences comprised within an SV40 viral vector. Minimizing sequence identity is particularly advantageous for eliminating homologous recombination during packaging process of the SV40 vectors. The expression cassette of the invention comprises:
a. a nucleic acid sequence coding for SV40 T-Ag;
b. a heterologous promoter, preferably an inducible promoter;
c. a heterologous termination signal; and
d. optionally additional operably linked control elements and/or selectable markers.

According to a particular and specifically preferred embodiment, the expression cassette of the invention comprises SV40 T-Ag encoding fragment corresponding to nucleotides 5179 to 2687 of the SV40 genome as denoted by GenBank Accession Number J20400, the inducible mouse metalloprotein promoter and a bovine growth hormone polyadenylation signal. In a preferred embodiment, this expression cassette is comprised within a plasmid. A preferred plasmid is the plasmid designated pUMTB.

Still further, the present invention provides for a method for the production of a complementation SV40 packaging cell line for in-trans complementation of viral T-antigen (T-Ag), which method comprises the steps of providing primate SV40 permissive or semi-permissive cells; transforming the cells with at least one expression cassette of the invention and optionally further transforming these cells with an additional expression cassette comprising a gene coding for a selectable marker; and selecting for transformed cells expressing the expression cassette of the invention.

The present invention further provides a process for the production of a preparation comprising recombinant T-Ag-deleted SV40 viral and/or pseudoviral vector and devoid of T-Ag containing recombinant SV40 viral and/or pseudoviral vector. This process, according to a preferred embodiment of this third aspect of the invention, comprises the steps of providing a complementation packaging cell line of the invention, infecting this cell line with an SV40 viral vector, or transfecting the cell line with vector DNA, culturing the infected cells under suitable conditions for permitting the production of said SV40 viral and pseudoviral vectors and harvesting the viruses or pseudoviruses. Virus harvesting and purification methods are exemplified in the Experimental procedures. However, it should be appreciated that different other purification methods may be applied and the disclosed methods should be considered only as non-limiting examples.

In those cases where the SV40 vector used for infecting or transfecting the complementation cell line does not contain late gene sequences, co-transfection of the viral vector and a helper vector carrying the late genes would be necessary.

Of particular interest is a process for the production of recombinant T-Ag deleted SV40 viral or pseudoviral vector according to the invention, wherein the complementation packaging cell line used is the COT18 cell line of the present invention (deposited under DSM Accession No. ACC2525).

The present invention provides a T-Ag deleted SV40 viral vector produced by the process of the invention. Moreover, the invention further provides preparation of T-Ag deleted SV40 viruses and pseudoviruses which are safe for medical use since they do not include recombinant viable viruses that carry and express the T-Ag.

The SV40 viral vector or pseudoviral vector produced by the process of the invention comprises an exogenous nucleic acid sequence. The nucleic acid sequence may be any one of DNA encoding an exogenous protein or peptide product, or encoding therapeutic RNA, or itself a therapeutic product. The therapeutic RNA may be antisense RNA, ribozyme RNA, iRNA (interference RNA) for targeted elimination of specific mRNA or any RNA, which inhibits or prevents the expression of undesired protein/s in any target cell.

According to a particularly preferred embodiment, the SV40 viral vector or pseudoviral vector produced by the process of the invention comprises exogenous nucleic acid selected from the group consisting of DNA encoding an exogenous protein or peptide product, or encoding a therapeutic RNA, or itself a therapeutic product, a DNA encoding an antisense RNA, ribozyme RNA, a DNA sequence encoding an iRNA (interference RNA) or any RNA or is itself DNA which inhibits or prevents the expression of undesired protein/s in any target cell, and optionally additional operably linked control elements.

More specifically, the therapeutic protein or peptide product encoded by exogenous nucleic acid sequence comprised within the SV40 viral vector or pseudoviral vector of the invention, may be at least one of a therapeutic protein or peptide product which is not expressed in a target cell. Alternatively, this product may be expressed in abnormally low amount, in defective form or is expressed in physiologically abnormal amount, in a target cell.

This therapeutic protein or peptide product may be according to a preferred embodiment at least one of an enzyme, a receptor, a membrane protein, a structural protein, a regulatory protein, a hormone, a synthetic protein or a peptide and a chimeric protein or a peptide.

Still further, the invention provides for a therapeutically safe preparation of any one of SV40 viral and pseudoviral vector according to the invention, prepared by the process as defined by the invention.

The present invention further provides a pharmaceutical composition comprising as an effective ingredient any one of SV40 viral and pseudoviral vector coding for a therapeutic protein or peptide product according to the invention or a safe preparation comprising the same, for the treatment of any pathological disorder in a subject in need.

A further aspect of the present invention relates to the use of the complementation cell line of the invention, for the preparation of a pharmaceutical composition. This composition comprises as an effective ingredient an SV40 viral vector or pseudoviral vector coding for a therapeutic protein or peptide product according to the invention or a safe preparation comprising the same, which is free of contaminating viable SV40 virus or SV40-like virus The composition according to the present invention is intended for ex vivo or in vivo transformation or transduction of cells for the ex vivo or in vivo treatment of a subject suffering from any pathological disorder.

### BRIEF DESCRIPTION OF THE INVENTION

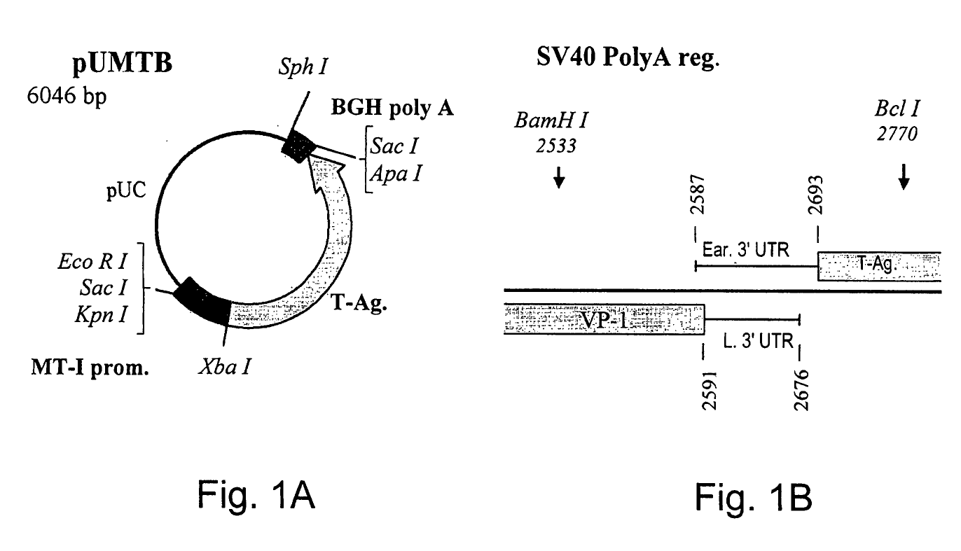

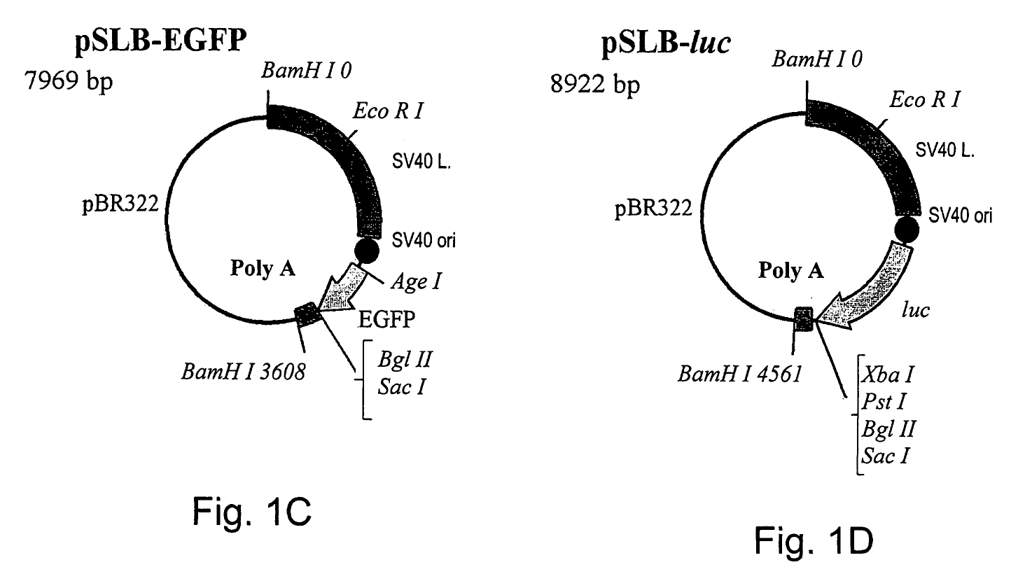

FIG. 1A-1D. Plasmid maps

1A: Plasmid pUMTB contains a pUC bacterial sequence backbone; approximately 650 bp of the mouse metallothionein promoter up to the Kpn I site (GenBank accession number X15128), T-antigen (T-Ag) coding sequence nt 5179-2687 (GenBank accession number J02400) and bovine growth hormone polyadenylation fragment, derived from plasmid pcDNA3 (Invitrogen) coordinates 984-1243.

1B: Physical map of the SV40 polyadenylation region. The vectors contain a fragment between the Bcl I site (coordinate 2533) and the BamH I site (coordinate 2770). The pUMTB construct carries a short (83 bp) overlap with the SV40 vectors, coordinates 2687-2770.

1C: Plasmid PSLB-EGFP contains the SV/GFP vector DNA inserted as a BamH I fragment into pBR332. T-antigen coordinates 5171-2770 is replaced by the EGFP gene, a Nhe I-Bgl II fragment derived from pEGFP-C1 (GenBank accession number U55763, coordinates 592 to 1340).

1D: Plasmid pSLB-luc is similar to pSLB-EGFP except that the luc gene fragment (Hind III to Xba I) derived from pGL3-basic (GenBank accession number U47295, coordinates 53 to 1742) replaces the T-antigen sequences.

Abbreviations: prom. (promoter), L. (late), reg. (region), Ear. (early), ori (origin of replication), UTR (un-translated region).

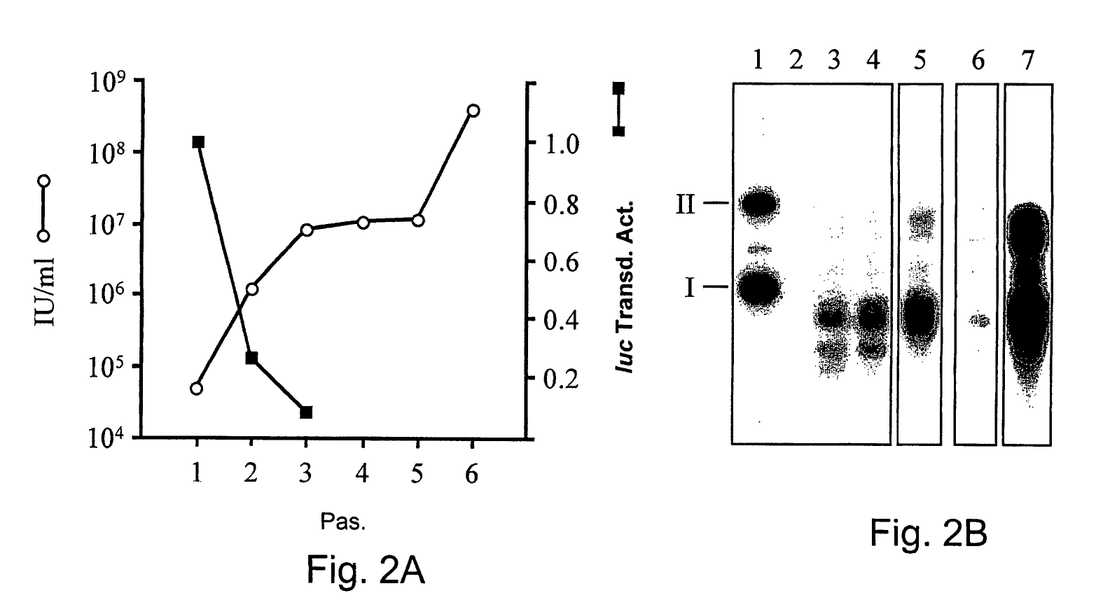

FIG. 2A-2B. Vector propagation on CMT4 cells

2A: SV/luc vector DNA was propagated by repeated passaging in CMT4 cells. The vector stocks were titered for IU and assayed for luc-transducing activity.

2B: Analysis of recombination products. Low molecular weight DNA from COS-1 and CV-1 cells infected with SV/luc was analyzed by Southern blotting. The blot was hybridized to a T-antigen specific probe (Hind III fragment of SV40 DNA, nucleotides 4002 to 5171). Lanes: 1, SV40 DNA marker. Form I (supercoiled) and form II (relaxed) of SV40 (5.3 kb) are marked on the left; 2, uninfected COS-1 cells; 3, COS-1 cells infected with 3^{rd}-passage vector; 4, COS-1 cells infected with 4^{th}-passage vector; 5, COS-1 cells infected with 6^{th}-passage vector; 6, CV-1 cells infected with 4^{th})-passage vector; 7, CV-1 cells infected with 6^{th}-passage vector. Blots were exposed to X-ray film at −70° C. with 2 intensifying screens for 3 hours (lanes 1-5) or 5 days (lanes 6-7).

Abbreviations: IU (infective units), Pas. (passage), Transd. (transducing), Act. (activity).

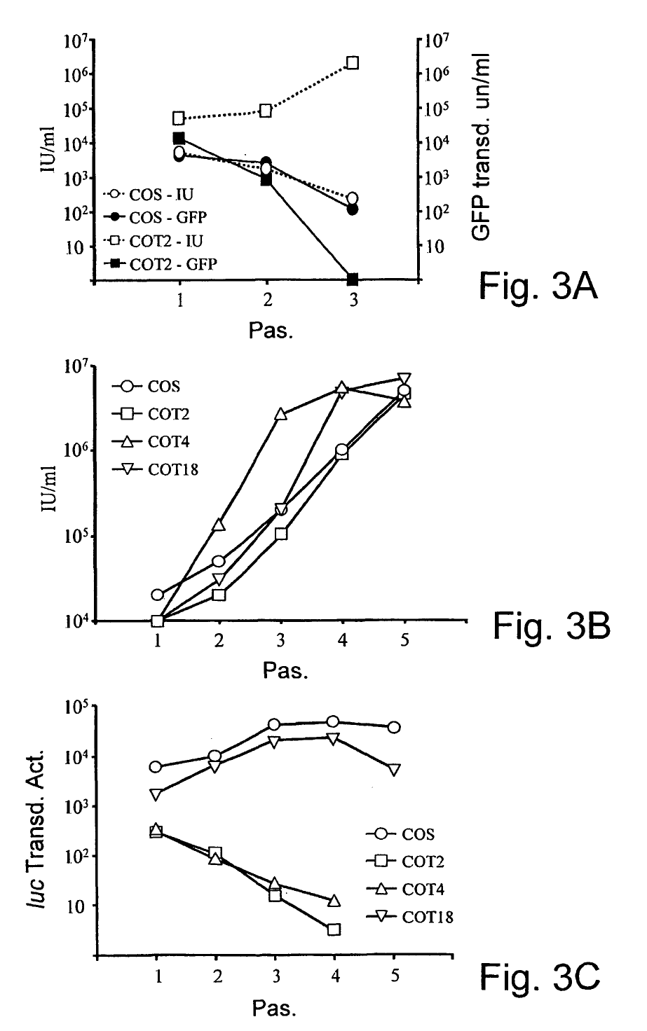

FIG. 3A-3C. Vector propagation in COT cell-lines

The vectors were analyzed for both IU titers and transducing activity.

3A: SV/GFP propagated on COT2 and COS-1 cell-lines were titered for IU and for GFP expressing cells.

3B: IU of SV/luc vectors stocks prepared in COS-1, COT2, COT4 and COT18 cell-lines.

3C: luc-transducing activity of the vector stocks shown in part B, was measured by infecting COS-1 cells with equal aliquots and assaying luc activity at 2 days post infection.

Abbreviations: IU (infective units), Pas. (passage), Transd. (transducing), Act. (activity), un. (units), GFP (green fluorescent protein).

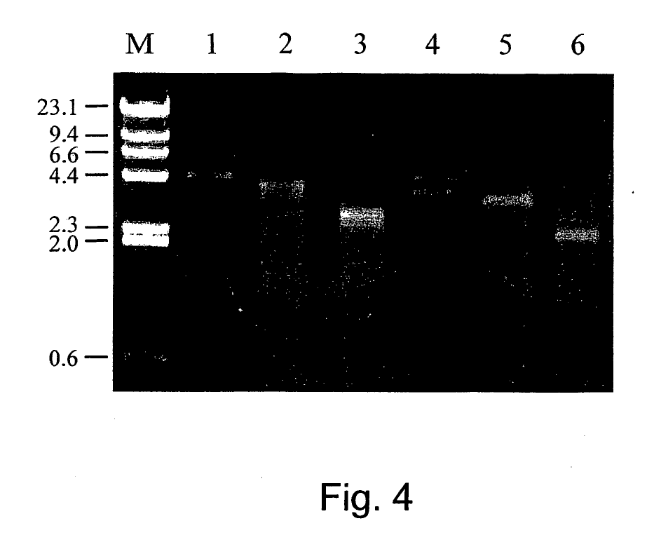

FIG. 4. Analysis of vector DNA

COT2 cells were infected with the specified vector stock and low molecular weight DNA was extracted 3 days post infection. The extracted vector DNA was linearized with EcoR I and electropherosed in parallel to the input vector DNA released from the shuttle plasmids by BamH I digestion. Lanes: M (marker), Hind III digested X DNA; 1, Input pSLB-luc digested with BamH I. The pBR322 fragment (4361 bp) and the excised SV/luc fragment (4561 bp) appear as a single band; 2, 5th-passage SV/luc DNA linearized with EcoR I; the two bands represent two different size deletions; 3, Uncut 5th-passage SV/luc DNA, showing the presence of heterogeneous size DNA; 4, Input pSLB-EGFP digested with BamH I. The pBR322 fragment is 4361 bp and the SV/GFP fragment is 3608 bp; 5, 3rd-passage SV/GFP DNA linearized with EcoR I, showing a smear of multiple bands slightly smaller than the input SV/GFP DNA; 6, Uncut 3rd-passage SV/GFP DNA showing a smear of multiple bands.

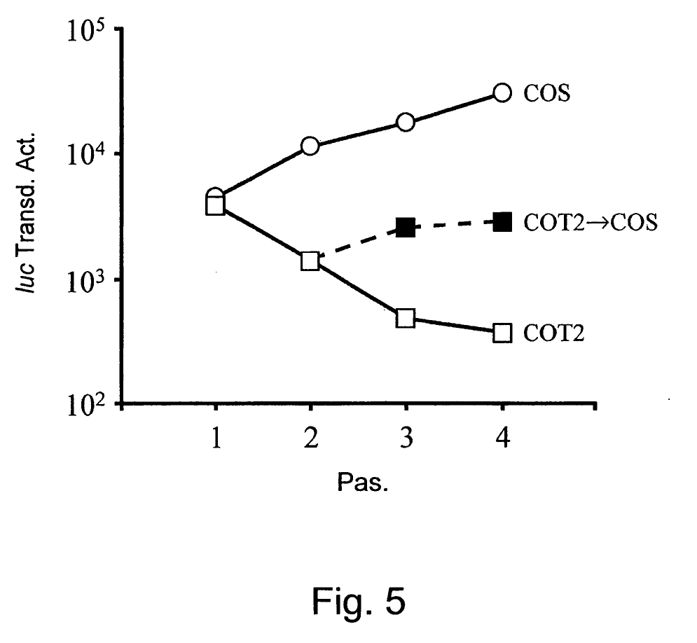

FIG. 5. The rate of generation of defective recombinant is cell-type dependent.

Transducing activity of SV/luc vectors propagated on COT2 and COS-1 cells. SV/luc vectors were passaged in COT2 cells. After the 2nd-passage, aliquots of vector stock were further passaged, in parallel, in both COT2 and COS-1 cells. In a parallel experiment SV/luc vectors were passaged continuously in COS-1 cells. luc-transducing activity was measured as in FIG. 3C.

Abbreviations: Pas. (passage), Transd. (transducing), Act. (activity).

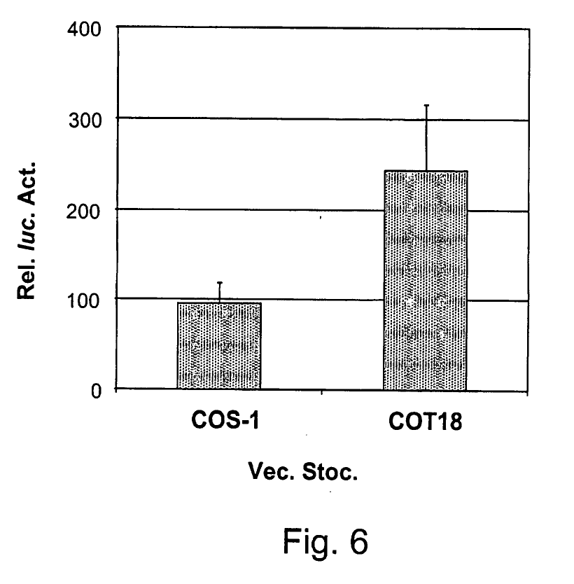

FIG. 6. Transduction of HuH-7 cells with SV/luc prepared using COS-1 or COT18

The histogram shows the relative luciferase activity measured as described in Example 5. Abbreviations: Rel. (relative) Luc. (luciferase), act. (activity), vec. (vector), sto. (stock).

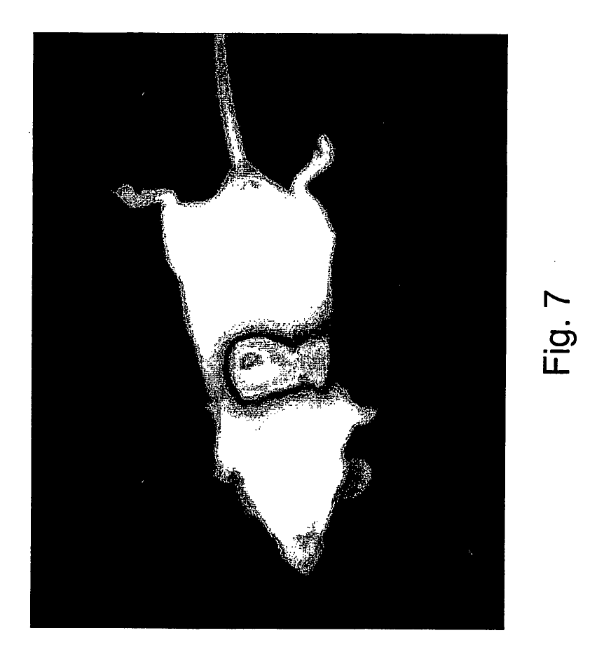

FIG. 7 In vivo targeting of viral vectors

Mouse injected with 1 ml of SV/luc prepared on COT18 cells, photographed 1 day post-injection.

### DETAILED DESCRIPTION OF THE INVENTION

A number of methods of the art of molecular biology are not detailed herein, as they are well known to the person of skill in the art. Such methods may include cloning, site-directed mutagenesis, expression of cDNAs, analysis of recombinant proteins or peptides, polymerase chain reaction (PCR), transfection of bacterial or mammalian cells, and the like. Textbooks describing such methods are e.g., Sambrook et al., Molecular Cloning A Laboratory Manual, Cold Spring Harbor Laboratory; ISBN: 0879693096, 1989, Current Protocols in Molecular Biology, by F. M. Ausubel, ISBN: 047150338X, John Wiley & Sons, Inc. 1988, and Short Protocols in Molecular Biology, by F. M. Ausubel et al. (eds.) 3rd ed. John Wiley & Sons; ISBN: 0471137812, 1995. These publications are incorporated herein in their entirety by reference. Furthermore, a number of immunological techniques are not in each instance described herein in detail, as they are well known to the person of skill in the art. See e.g., Current Protocols in Immunology, Coligan et al. (eds), John Wiley & Sons. Inc., New York, N.Y. Vector propagation and methods of titration are described in detail below.

Simian virus 40 (SV40) was discovered in 1960 as a contaminant in polio vaccines prepared from rhesus monkey kidney cell cultures. It was found to cause tumors when injected into newborn hamsters. The genome of SV40 is a double-stranded, circular DNA of about 5000 bases encoding large (708 amino acids) and small T antigens (174 amino acids), agnoprotein and the structural proteins VP1, VP2 and VP3. The respective size of these molecules is 362, 352 and 234 amino acids.

Large T antigen, one of the early proteins, plays a critical role in replication and late gene expression and is modified in a number of ways, including N-terminal acetylation, phosphorylation, poly-ADP ribosylation, glycosylation and acylation, as described in WO 99/27123. The other T antigen is produced by splicing of the large T transcript. The corresponding small T protein is not strictly required for infection, but it plays a role in the accumulation of viral DNA. DNA replication is controlled, to an extent, by a genetically defined core region that includes the viral origin of replication. The SV40 element is about 66 bp in length and has sub-sequences of AT motifs, GC motifs and an inverted repeat of 14 bp on the early gene side. Large T-antigen is required for initiation of DNA replication, and this protein has been shown to bind in the vicinity of the origin. It also has ATPase, adenylating helicase activities.

After viral replication begins, late region expression initiates. The transcripts are overlapping and, in some respect, reflect different reading frames (VP1 and VP2/3). Late expression initiates in the same general region as early expression, but in the opposite direction. The virion proteins are synthesized in the cytoplasm and transported to the nucleus where they enter as a complex. Virion assembly also takes place in the nucleus, followed by lysis and release of the infectious virus particles.

Simian virus 40 vectors are efficient vehicles for gene delivery to a variety of human target cells, especially hematopoietic and hepatic cells. Contamination by replication-competent virus plagues viral vector stocks including retroviral, adenoviral and adeno-associated viral vectors. The inherent instability of some viral vectors hinders the ability to precisely predict the nature of contaminants in vector stocks, thus raising serious safety concerns. It has been found by the present inventors that SV40 vector stocks propagated on COS-1 and CMT4 cells were sometimes contaminated with plaque-forming SV40 at a ratio of >1 PFU (plaque forming units) per 105 IU (infective units). The rate of recombination leading to replication-competent SV40 was reported to be even higher in COS-7 than in COS-1 cells [Jasin, ibid., (1985)]. The COT cell-lines of the invention were therefore constructed in an effort to eliminate such contamination by reducing the regions of identity between the packaging construct used for formation of the complementation packaging cell line, and the vector sequences below the length required for homologous recombination in mammalian cells. As described in the following Examples, this strategy indeed has eliminated the emergence of replication-competent SV40.

Thus, in a first aspect the present invention relates to a complementation SV40 packaging cell line for in-trans complementation of viral T-antigen (T-Ag). Which cell line eliminates the generation of viable recombinant virus carrying the T-Ag gene. This cell line is transformed with at least one expression cassette having minimal (between 0 to 300 and preferably less than 100 bp) sequence identity to SV40 sequences comprised within SV40 viral vectors. Minimizing sequence identity is particularly required and advantageous for eliminating homologous recombination in packaging process of the SV40 vectors. The expression cassette introduced into the complementation cells of the invention comprises SV40 T-Ag, heterologous promoter, heterologous termination signal and optionally additional operably linked control elements and/or selectable markers. Minimal sequence identity is meant that SV40 sequences comprised within the cell line and the viral vectors, share minimal sequence, e.g., between 0 to 300, preferably, 0 to 200 and most preferably, less than 100 nucleotides.

In context of the present invention, the term “complementation cell line” refers to a eukaryotic cell permissive or semi-permissive for SV40 propagation and capable of providing in-trans the function(s) for which an SV40 vector is defective. In other words, it is capable of producing the protein or proteins needed for replication and indirectly for encapsidation of said SV40 vector. Naturally, said protein may be modified by mutation, deletion and/or addition of nucleotides, as long as these modifications do not impair its capacity for complementation.

The complementation line according to the invention may be derived either from an immortalized cell line capable of dividing indefinitely, or from a primary line. In accordance with the objectives pursued by the present invention, a complementation line is useful for the replication and/or for encapsidation of any defective SV40 vector, and especially a T-Ag deleted SV40 vector. Thus, when the term “deleted SV40 vector” or “defective SV40 vector” is used below, it should be understood to refer to any T-Ag deleted vector, and is not limited to the T-Ag deleted vector of the present invention.

These SV40 T-Ag deleted viral vectors may be, according to the invention, vectors comprising nucleic acid sequences coding for SV40 late proteins as well as SV40 ori. The transgene introduced to these vectors has limited size. Alternatively, the viral vectors may comprise limited SV40 sequences, and may be only the ori without any late proteins. These vectors may be packaged by the cells of the invention only in the presence of a helper vector carrying the late proteins, and therefore are referred herein as SV40 pseudoviral vectors. These vectors may carry much larger transgene.

An example of a helper that will supply the late genes is SLT3 [Oppenheim, A. and Peleg, A. Gene 77, 79-86 (1989)].

Of particular interest is the T-Ag which serves as a complementation element for the T-Ag deleted SV40 vectors. “Complementation element” is understood to mean a nucleic acid comprising at least the portion of the SV40 genome used in the context of the present invention. It is to be appreciated that any fragment as well as any mutant of the SV40 T-Ag sequence that supports any stage of the SV40 life cycle, and particularly replication and also packaging, is contemplated within the scope of the present invention. Preferably, the part of T-antigen that is required to support vector DNA replication. The complementation element can be inserted into a vector, for example of the plasmid or viral type, and preferably a plasmid. The case where the complementation element is integrated in the genome of a complementation line according to the invention will nevertheless be mostly preferred. In order to facilitate integration of such expression vector to the cell genome, the cells were transformed using linearized expression cassettes as detailed in the Examples. The methods for introducing a vector or a nucleic acid into a cell line, and possibly of integrating it in the genome of a cell, constitute conventional techniques well known to a person skilled in the art, as do the vectors which can be used for such purposes. The complementation element may be introduced into a complementation line according to the invention beforehand or concomitantly with a defective SV40 vector, preferably before infection.

As used herein, the term “nucleic acid” refers to polynucleotides such as deoxyribonucleic acid (DNA), and cDNA. The terms should also be understood to include, as equivalents, analogs of either RNA or DNA made from nucleotide analogs, and, as applicable to the embodiment being described.

The term “operably linked” is used herein for indicating that a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Accordingly, the term control and regulatory elements includes promoters, terminators, enhancers, introns and other expression control elements. Such regulatory elements are described in Goeddel [Goeddel, Gene Expression Technology: Methods in Enzymology 185, Academic Press, San Diego, Calif. (1990)]. For instance, any of a wide variety of expression control sequences, which are sequences that control the expression of a DNA sequence when operatively linked to it, may be used in the expression cassette of the invention to express DNA sequences encoding the T-Ag.

Cell lines according to the invention can be constructed in various ways. In general, they are prepared by the transformation of a cell or cell culture with a plasmid carrying an expression cassette comprising nucleic acid fragment comprising the SV40 T-Ag encoding region under the control of a promoter functional in said cell. Construction of suitable plasmids containing the expression cassette comprising the desired SV40 T-Ag, heterologous promoter and/or genetic elements system employs standard ligation techniques. Isolated plasmids or nucleotide sequences are cleaved, tailored, and re-ligated in the form desired to form the plasmid required. Plasmid vectors are usually amplified in bacterial hosts prior to transfection, the inducible promoter or genetic elements in a vector including one or more phenotypic selectable markers and origin of replication to ensure amplification within a bacterial host.

Transfection of the cells can be carried out by any technique known to persons skilled in the art, and especially in the presence of calcium phosphate, by lipofection or by electroporation. In a specific embodiment, the plasmid used also carries a marker gene, which makes it possible to identify and to select the transformed cells. This may be, for example, any gene for resistance to an antibiotic (e.g. geneticin G418, or hygromycin). The marker gene may also be carried by a separate construct, co-transfected with the plasmid.

Therefore, in a preferred embodiment the packaging cell lines of the invention are optionally further transformed with an additional expression cassette comprising a gene coding for a selectable marker.

A variety of selectable markers can be incorporated into any construct. For example, a selectable marker which confers a selectable phenotype such as drug resistance, nutritional auxotrophy, resistance to a cytotoxic agent or expression of a surface protein, can be used. Selectable marker genes which can be used include neo, gpt, dhfr, ada, pac, hyg, CAD, KAN, URA3, HIS3 and LEU2. The selectable phenotype conferred makes it possible to identify and isolate recipient cells. Amplifiable genes encoding selectable markers (e.g., ada, dhfr and the mutifunctional CAD gene which encodes carbamyl phosphate synthase, aspartate transcarbamylase, and dihydro-orotase) have the added characteristic that they enable the selection of cells containing amplified copies of the selectable marker inserted into the genome. This feature provides a mechanism for significantly increasing the copy number of an adjacent or linked gene for which amplification is desirable.

After transfection and selection for the marker gene, the cells obtained can be selected for their capacity to transcomplement SV40 vectors lacking the T-Ag region. For that, various T-Ag deleted SV40 vectors can be used and especially vectors carrying a reporter gene such as SV/GFP and SV/LUC as described in Examples 3 and 4.

According to the invention, a complementation line is intended to complement in-trans a defective SV40 vector for the T-Ag function. Such a line has the advantage of eliminating the risks of recombination since, contrary to the conventional COS and CMT cells and more particularly, the COS-1, COS-7 and CMT4 lines, the complementation cell line of the present invention carries only minimal sequence identity to the SV40 sequences comprised within the SV40 vectors. Such limited identity is below the minimum required for homologous recombination.

Thus, in a particularly preferred embodiment, in addition to the T-Ag sequence, the complementation cells of the invention have minimal sequence identity to the SV40 viral vectors. According to the invention, the sequence identity to the SV40 viral vectors is below the identity required for efficient homologous recombination of the SV40 sequences comprised within an SV40 viral vectors and sequences of said cell line. According to a preferred embodiment, the SV40 sequences comprised within the cells and the SV40 sequences comprised within said SV40 viral vector share a sequence identity of between 0 and about 300 nucleotides, preferably, between 0 to about 250 nucleotides, more preferably between 0 to about 100 nucleotides. Most preferably, between 0 to about 10 and especially most preferably, the complementation cells of the invention do not contain any sequence identity to the SV40 sequence comprised within the SV40 vectors.

By the term “share” is meant that each (the viral vector and the packaging cell line) contain the same identical nucleotide sequence.

In a particular specific embodiment, the SV40 sequences comprised within the cells and the SV40 viral vector share 83 nucleotides corresponding to nucleotide 2770 to 2687 of the SV40 genome as denoted by GenBank Accession Number J20400. By the term “share” is meant that each (the viral vector and the packaging cell line) contain the same identical 83 nucleotide sequence. This can be further reduced by in vitro mutagenesis to less than 10, by mutating the primary DNA sequence of the 83 nucleotides without changing the amino acids coded, using the degeneration of the code.

An alternative possibility to completely eliminate sequence identity between the vector and the complementing cell line is by deleting from the vector nucleotides 2693 to 2770. This can be readily accomplished using modern molecular technology (cloning by PCR).

Moreover, according to a variant of this embodiment, the complementation cell line according to the invention carries the portion of the SV40 genome coding for the early protein T-Ag placed under the transcriptional control of a suitable heterologous promoter which is functional in said complementation line. This promoter can be isolated from any eukaryotic or viral gene, but the use of an SV40 promoter of an early region, however, should be avoided. It should be appreciated that the use of heterologous promoter enables the reduction of overlapping identical sequences between the SV40 vectors and the complementation cell line of the invention. The promoter may be a constitutive promoter, as examples, the TK-HSV-1 gene and murine PKG gene promoters may be mentioned.

Alternatively and preferably, the promoter selected may be regulable and advantageously inducible by a protein or any other element which trans-activates transcription. It can be a promoter isolated from a naturally inducible gene or any promoter modified by the addition of activating sequences (or UAS, standing for upstream activating sequence) responding to said trans-activating protein. More specially, it is preferable to use a promoter which is inducible by heavy metals, and most preferably the inducible metalloprotein promoter of a mouse.

A “promoter” refers to a DNA sequence recognized by the synthetic machinery of the host cell, or introduced synthetic machinery, that is required to initiate the specific transcription of a gene. The phrase “under transcriptional control” means that the promoter is in the correct location in relation to the polynucleotide to control RNA polymerase initiation and expression of the polynucleotide.

Examples for other different inducible promoters that may be used for expression of the T-Ag complementing element according to the invention are disclosed hereinafter.

In a further preferred embodiment, the T-Ag expressed in the packaging cell line of the invention is placed under the control of a heterologous termination signal. More particularly, the heterologous termination signal or sequence may be a bovine growth hormone polyadenylation signal. It should be appreciated that the use of heterologous termination sequence enables the reduction of overlapping identical sequences between the SV40 vectors and the complementation cell line of the invention.

In a particularly preferred specific embodiment, the SV40 T-Ag region expressed by the packaging cell line of the invention is a nucleic acid fragment corresponding to nucleotides 5179 to 2687 of the SV40 genome as denoted by GenBank Accession Number J20400. It should be noted that this fragment contains the T-Ag coding sequence (5163-2694) as well ad additional flanking sequences.

The expression cassette according to one embodiment is comprised within a plasmid, preferably the plasmid designated pUMTB. Thus, in yet another specifically preferred embodiment the packaging cell line of the invention is transformed by the pUMTB plasmid. This plasmid comprises an expression cassette comprising SV40 T-Ag fragment corresponding to nucleotides 5179 to 2687 of the SV40 genome, expressed under the control of the inducible mouse metalloprotein promoter and a bovine growth hormone polyadenylation signal.

Thus, the complementation line according to the invention comprises, in particular, the portion of the genome of SV40 extending from nucleotide 5179 to nucleotide 2687 of the sequence of the SV40 genome as disclosed in the GenBank data bank under the reference J02400. It is to be mentioned that nucleotides 2770 to 2687 of the SV40 genome are the 83 nucleotides overlapping with the SV40 vectors.

It is to be appreciated that any fragment as well as any mutant of the SV40 T-Ag sequence that supports any stage of the SV40 life cycle, and particularly replication and also packaging, is contemplated within the scope of the present invention.

According to a preferred embodiment, a complementation line of the invention may contain a complementation element comprising, in addition, a gene coding for a selectable marker permitting the detection and isolation of the cells containing it. In the context of the present invention, this gene can be any gene coding for a selectable marker, such genes being generally known to a person skilled in the art and some of which are mentioned above. Advantageously, a gene for resistance to an antibiotic, and preferably the gene coding for neomycin resistance conferring resistance to neomycin (G418) is being used in the present invention.

According to another specific embodiment, the packaging cell line of the invention is further transformed with additional expression cassette comprising a gene coding for a selectable marker. This cassette is comprised within a plasmid, which according to a specific embodiment is the pLN, containing a sequence coding for neomycine resistance.

Thus, in a specifically preferred embodiment, the packaging cell line of the invention is co-transformed by the plasmids pUMTB and pLN.

According to a particularly preferred embodiment, a complementation line according to the invention is derived from a cell line which is acceptable from a pharmaceutical standpoint. “Cell line which is acceptable from a pharmaceutical standpoint” is understood to mean a cell line which is characterized (whose origin and history are known), having known pathogenic properties. These may be established cell lines or primary cultures and/or which have already been used for the large-scale production of products intended for human use (assembly of batches for advanced clinical trials or of commercial batches). Such lines are available from bodies such as the ATCC. Therefore, the complementation cell line may be derived from any primate cell line permissive or semi-permissive for SV40. In this connection, the following cell lines may be mentioned: Vero African Green monkey kidney, CV-1, Hela which derived from a human epithelial carcinoma and BSC-1.

Alternatively, a complementation line according to the invention can be derived from primary cells, and in particular from cells taken from a primate embryos. “Primary cells” are cells that have been harvested from the tissue of an organ.

According to a particularly preferred embodiment, cells according to the invention are derived from the Vero and CV-1 cell line. In this regard, particularly advantageous results have been obtained with cells of the line CV-1 which are transformed with the plasmid pUMTB. The present invention also provides plasmids comprising the expression cassette of the invention which comprises T-Ag encoding sequence of an SV40 genome under the control of an inducible promoter. A preferred plasmid of this type is plasmid pUMTB as described in Experimental procedures and also disclosed by FIG. 1A.

In a particularly preferred embodiment, the packaging cell line of the invention is derived from CV-1 cell line.

Of particular interest are the CV-1 derived packaging cell lines selected from the group consisting of COT2, COT4 and COT18. Most preferred CV-1 derived cell line is the COT18. This cell line was deposited under DSM Accession No. ACC2525.

It is to be appreciated that “complementation cells or cell lines or lines” or “packaging cells or cell line or lines” are terms used interchangeably herein. It is to be understood that such terms refer not only to the particular subject cells but to the progeny or potential progeny of such a cell. Because certain modifications may occur in a succeeding generation due to either mutation or environmental influences, such progeny may not, in fact, be identical to the parent cell, but are still included within the scope of the term as used herein.

The COT18 complementation cell-line constructed and characterized in the present invention offers a means for the production of safe SV40 viral vector stocks or preparations, at a titer of at least 10⁷-10⁸ IU/ml.

Moreover, it is to be appreciated that the complementation cell lines of the present invention, that were prepared by the method described herein and can be use for production of safe SV40 vectors, may be used also as a powerful tool for basic research of different biological and physiological aspects on SV40.

Furthermore, the complementation cell line of the invention can be used for mass production of a desired protein. Alternatively, these cells may be used for screening of a cDNA library in SV40 packaged in vivo to identify proteins having specific properties, that could affect specific tissues such as HIV infected cells or cancer cells. High throughput screening assay could allow isolation of specific clones with therapeutic properties (or any other desired properties, for example, for biotech purpose).

In a second aspect, the present invention relates to an expression cassette for the production of a complementation SV40 packaging cell line. This expression cassette has minimal sequence identity to SV40 sequences comprised within an SV40 viral vector. Minimizing sequence identity is particularly required for eliminating homologous recombination in preparation process of the SV40 vectors. The expression cassette of the invention comprises:
a. a nucleic acid sequence coding for SV40 T-Ag;
b. a heterologous promoter;
c. a heterologous termination signal, preferably, polyadenylation sequence; and
d. optionally additional operably linked control elements and/or selectable markers.

The expression cassette for the production of the complementing T-Ag molecules according to the invention may be included in vectors such as plasmids, phagemids or other vectors. “Vectors”, as used herein, encompass plasmids, viruses, bacteriophage, integratable DNA fragments, and other vehicles, which enable the integration of DNA fragments into the genome of the host. Expression cassettes are typically self-replicating DNA or RNA constructs containing the desired nucleic acid sequence or its fragments, and operably linked genetic control elements that are recognized in a suitable host cell and effect expression of the desired genes within the host. Generally, the genetic control elements can include a prokaryotic promoter system or a eukaryotic promoter expression control system. Such system typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression cassettes or vectors usually contain an origin of replication that allows the vector to replicate independently of the host cell.

A DNA sequence may be recombined with a DNA vector in accordance with conventional techniques. Conventional techniques include techniques such as: blunt-ended or staggered-ended ligation, restriction enzyme digestion to provide appropriate termin, filling of cohesive ends as appropriate, alkaline phosphatase treatment to avoid undesirable joining, and ligation with appropriate ligases, or PCR of the vector and the insert. Techniques for such cloning manipulations are well known in the art. A DNA molecule can be capable of being expressed if it contains transcriptional and translational regulatory elements. Such regulatory elements should be operably linked to the encoding nucleotide sequences.

For cloning purpose, a vector may additionally include appropriate restriction sites, antibiotic resistance or other markers for selection of vector containing cells. Plasmids are the most commonly used form of vector and are specifically preferred according to the invention, but other forms of vectors which serves an equivalent function and which are, or become, known in the art are suitable for use herein. See, e.g., Pouwels et al. [Cloning Vectors: a Laboratory Manual (1985 and supplements), Elsevier, N.Y.] and Rodriquez, et al. (eds.) [Vectors: a Survey of Molecular Cloning Vectors and their Uses, Buttersworth, Boston, Mass. (1988)], which are incorporated herein by reference.

In general, such vectors contain in addition specific genes, which are capable of providing phenotypic selection in transformed cells. The use of prokaryotic and eukaryotic expression vectors (plasmids) to express the expression cassette comprising the SV40 T-Ag sequence according to the present invention are also contemplated. These vectors may further contain tagging sequences which are capable of providing convenient isolation or detection of the desired expressed sequences. Such tagging sequences are well known in the art and include for example FLAG, HA, His-6 (six histidine) and the myc tag.

The vector is introduced into a host cell by methods known to those of skilled in the art. Introduction of the vector into the host cell can be accomplished by any method that introduces the construct into the cell, including, for example, transfections by calcium phosphate precipitation, lipofection, microinjection, electroporation or transformation. See, e.g., Current Protocols in Molecular Biology, Ausuble, F. M., ed., John Wiley & Sons, N.Y. (1989). As used herein, the term “transfection” means the introduction of a nucleic acid, e.g., the DNA construct or an expression vector, into a recipient cells by transient or stable nucleic acid-mediated gene transfer, preferably, stable. “Transformation”, as used herein, refers to a process in which a cell's genotype is changed as a result of the cellular uptake of exogenous DNA or RNA.

In a particular embodiment, it will be desired to employ an inducible promoter or promoter system to control expression of the T-Ag region included within the complementation cells of the present invention. Selection of a promoter that is regulated in response to specific signals permits inducible expression of the gene product. Several inducible promoter systems are available for production of viral vectors where the transgene product may be toxic.

Inducible promoters and inducible genetic elements are known in the art and can be derived from viral or mammalian genomes. Numerous examples of inducible promoters are known in the art:

One possible inducible system is to construct a fusion product between the SV40 T-Ag sequences and a steroid hormone receptor. This has been shown to result in steroid inducible function of the fusion partner.

The ecdysone system (Invitrogen Carlsbad, Calif.) is another example for an inducible system. This system is designed to allow regulated expression of a gene of interest in mammalian cells. It consists of a tightly regulated expression mechanism that allows virtually no basal level expression of the transgene, but over 200-fold inducibility. The system is based on the heterodimeric ecdysone receptor of Drosophila, and when ecdysone or an analog such as muristerone A binds to the receptor, the receptor activates a promoter to turn on expression of the downstream transgene high levels of mRNA transcripts are attained. In this system, both monomers of the heterodimeric receptor are constitutively expressed from one vector, whereas the ecdysone-response promoter, which drives expression of the gene of interest, is on another plasmid. Engineering of this type of system into the expression vector of the invention would therefore be useful.

Another inducible system that would be useful is the Tet-Off™ or Tet-On™ system (Clontech, Palo Alto, Calif.) originally developed by Gossen and Bujard [Gossen and Bujard, Proc. Natl. Acad. Sci. USA, 89:5547-5551 (1992); Gossen et al., Science, 268:1766-1769 (1995)]. This system also allows high levels of gene expression to be regulated in response to tetracycline or tetracycline derivatives such as doxycycline. In the Tet-On™ system, gene expression is turned on in the presence of doxycycline, whereas in the Tet-Off™ system, gene expression is turned on in the absence of doxycycline. These systems are based on two regulatory elements derived from the tetracycline resistance operon of E. coli. The gene of interest (preferably the T-Ag according to the invention) may be cloned into a plasmid behind a promoter that has tetracycline-responsive elements present in it. A second plasmid contains a regulatory element called the tetracycline-controlled trans-activator, which is composed, in the Tet-Off™ system, of the VP16 domain from the herpes simplex virus and the wild-type tetracycline repressor. Thus in the absence of doxycycline, transcription is constitutively on. In the Tet-On™ system, the tetracycline repressor is not wild type and in the presence of doxycycline activates transcription.

Another inducible promoter system is disclosed in WO 98/21350. The promoter region of advenovirus E4 was replaced with a synthetic promoter composed of a minimal TATA box and five consensus 17-mer GAL4-binding site elements (GAL4/TATA). Since most mammalian cells express no GAL4-like activity, a synthetic GAL4-responsive promoter containing GAL4-binding sites and a TATA box should have no or extremely low basal activity in the absence of a GAL4 transactivator, and high activity in its presence. GAL4 is a transcriptional activator derived from yeast, that when fused to a highly acidic portion of the herpes simplex virus protein VP16, is a very potent activator of transcription [Sadowski et al., Nature, 335:563-564 (1988)]. Thus, genes that have GAL4 binding sites in their promoter regions are highly activated by the introduction of the GAL4-VP16 fusion protein. This technology could be applied for expression of genes in the complementation cell line, particularly, SV40 T-Ag.

Examples for other inducible promoters are the mouse mammary tumor virus (MMTV) promoter, induced by glucocorticoid [Beato, M, et al., J. Steroid Biochem, 27:9-14 (1987)], the lac repressor-lac operator inducible promoter system, which is based on the DNA binding protein namely lac repressor (lacI), and the lac operator (lacO), has been shown to function in mammalian cells [Brown, M, et al., Cell, 49:603-12 (1987)]. A most preferred inducible promoter may be the metallothionein promoter which is inducible by heavy metals [Mayo, K E, et al., Cell, 29:99-108 (1982)] and has been used in the present invention.

According to a particular and specifically preferred embodiment, the expression cassette of the invention comprises SV40 T-Ag encoding sequence fragment corresponding to nucleotides 5179 to 2687 of the SV40 genome, the inducible mouse metalloprotein promoter and a bovine growth hormone polyadenylation signal. In a preferred embodiment, this expression cassette is comprised within a plasmid. The plasmid comprising the expression cassette is designated pUMTB.

Still further, the invention provides for a method for the production of a complementation SV40 packaging cell line for in-trans complementation of viral T-antigen (T-Ag), which method comprises the steps of:
a. providing primate SV40 permissive or semi-permissive cells;
b. transforming said cells with at least one expression cassette as defined by the invention and optionally further transforming said cells with an additional expression cassette comprising a gene coding for a selectable marker; and
c. selecting for transformed cells expressing the expression cassette as defined by the invention.

The present invention further provides a process for the production of a substantially pure preparation comprising recombinant T-Ag-deleted SV40 viral and/or pseudoviral vector. By substantially pure is meant a preparation devoid of T-Ag containing recombinant SV40 viral and/or pseudoviral vector.

This process, according to a preferred embodiment of this third aspect of the invention, comprises the steps of providing a complementation packaging cell line of the invention, infecting this cell line with an SV40 viral vector or transfecting with vector DNA, culturing the infected cells under suitable conditions for permitting the production of said SV40 viral vectors and harvesting the viruses.

“Suitable culturing conditions” include as a non-limiting example, induction of the inducible promoter that would result in efficient expression for T-Ag, required for replication of the SV40 viral vector. If according to a preferred embodiment, the metalloprotein inducible promoter is being used, addition of heavy metals such as Zn⁺⁺ and Cd⁺⁺ to the cell culture medium will induce transcription of the T-Ag.

Of particular interest is a process for the production of recombinant T-Ag deleted SV40 viral vector as well as pseudoviral vectors according to the invention, wherein the complementation packaging cell line used is the COT18 of the present invention.

The present invention further provides a T-Ag deleted SV40 viral vectors produced by the process of the invention.

In T-Ag deleted SV40 viral vectors the coding regions have been replaced with transgene sequences and, therefore, a number of functions must be supplied in trans. In this particular instance, SV40 “early” functions are needed to facilitate the replication of the SV40 vector. Primarily, these functions derive from the SV40 T antigen. Thus, the invention's complementation cell line that provides an SV40 large T antigen is required and would enable replication as well as assembly of recombinant SV40 viral vectors.

It is to be appreciated that any fragment as well as any mutant of the SV40 T-Ag sequence that supports any stage of the SV40 life cycle, and particularly replication and also packaging, is contemplated within the scope of the present invention.

In case that the SV40 vector used contains only the SV40 origin of replication, the complementation cell line of the invention supports only the replication of such vector. These viral vectors are advantageous since they may comprise a larger transgene replacing the late genes. In this case a helper vector would be required to supply functions that support the assembly of such SV40 vector into SV40 pseudoviruses. These functions must be supplied in trans and include late gene products such as capsid proteins. A non limiting example of such helper vector is the SLT3 as disclosed in Oppenheim et al. [Oppenheim and Peleg, ibid. (1989)]

The recombinant T-Ag deleted SV40 vectors and specifically, the therapeutic preparation which comprises the same, possess properties which are particularly attractive for use in gene therapy. These preparations and vectors combine favorable properties as regards infection, safety (the risks of generation of viable SV40 by homlogous recombination is eliminated; immune and/or inflammatory reaction are eliminated) and strong capacity to transfer genes. Furthermore, the complementation cells and cell lines of the invention allow the production of viral stocks which are free of replication competent contaminating particles (RCA). Thus, the results presented in the Examples show the construction and the production, by the complementation lines of the invention, of T-Ag deleted SV40 vectors free of RCA. In particular, the appearance of T-Ag⁺ replicative contaminating particles during the production and packaging of the recombinant SV40 vectors according to the invention is hardly possible with the cells and cell lines of the invention since these cells contain almost no SV40 overlapping sequences on either side of the T-Ag region integrated into the genome of the cell.

According to a particularly preferred embodiment, the SV40 viral or pseudoviral vector produced by the process of the invention comprises an exogenous nucleic acid sequence. The exogenous nucleic acid may be selected from the group consisting of an exogenous DNA encoding an exogenous protein or peptide product, or encoding a therapeutic RNA, or itself a therapeutic product, a DNA encoding an antisense RNA, ribozyme RNA, a DNA sequence encoding an iRNA (interference RNA) for targeted elimination of specific mRNA or any RNA or DNA which inhibits or prevents the expression of undesired protein/s in any target cell.

More specifically, the therapeutic protein or peptide product encoded by the exogenous nucleic acid sequence comprised within the SV40 viral or pseudoviral vector or the safe preparations of the invention may be at least one of a therapeutic protein or peptide which is not expressed. Alternatively, this product may be expressed in abnormally low amount, in defective form or is expressed in physiologically abnormal amount, in a target cell.

The therapeutic protein or peptide product can be any protein of interest, such as an enzyme, a receptor, a membrane protein, a transporter, a structural protein, a regulatory protein or a hormone. Of particular interest are proteins which are missing or defective in patients suffering from genetic disorders.

Therapeutic products which are encoded by any nucleic acid sequences may be packaged and expressed by the SV40 viral or the pseudoviral vectors of the invention. Therapeutic products which are encoded by nucleic acid sequences smaller than about 2.2 Kb may be packaged and expressed by the SV40 viral vectors of the invention. These viral vectors comprising the late gene and therefore can carry only small transgenes. However, the SV40 pseudoviral vectors can comprise large transgenes due to deletion of their late genes. As a non-limiting examples of such transgenes that may serve as therapeutic products encoded by the SV40 viral and/or pseudoviral vectors of the invention are enzymes, e.g., adenosine deaminase (ADA), glucocerebrosidase (GC), hexoaminidase and the enzyme bilirubin-uridine 5′-diphosphate-glucuronosyl-transferase (BUGT) that is missing in patients with Crigler-Najjar syndrome; receptors, e.g. low density lipoprotein receptor (LDL receptor) and IL-6 receptor; cystic fibrosis transmembrane conductance regulator (CFTR) and multidrug resistance (MDR); regulatory proteins, e.g. p53 and retinoblastoma (Rb); hormones, e.g. insulin, growth hormone; and growth factors, e.g. IL-2 and IL-6 structural proteins, e.g. APO A-I and a single chain variable fragment (ScFv) against critical proteins of infectious agents (i.e. HIV integrase).

In a particular embodiment, the therapeutic protein is one which is lacking or is defective in hemopoietic disorders such as β-thalassemia, α-thalassemia, anemias due to deficiencies in red blood cell cytoskeletal or membrane proteins or enzymes, deficiencies in heme synthesis enzymes and deficiencies in erythroid transcription factors. Thus, in this particular embodiment the therapeutic protein is any one of α-globin, β-globin or γ-globin, preferably β-globin, which is lacking or defective in β-thalassemia and related diseases.

In a further preferred embodiment, the preparations, SV40 viral or pseudoviral vectors of the invention comprise therapeutic exogenous nucleic acid sequence, which is the human MDR1 gene. Enhancement of drug resistance is particularly important in connection with conferring resistance of the bone marrow cells to high dose chemotherapy. This mammalian gene confers resistance against variety of drugs such as colchicine, vinblastine, adriamycin and others.

In yet another specific embodiment, the therapeutic protein encoded by the exogenous nucleic acid sequence comprised within the SV40 viral and pseudoviral vectors according to the invention may be glucocerebrosidase. This enzyme is defective in Gaucher disease, and therefore overexpression of this enzyme by the pseudovirus or virus prepared using the complementation cell line of the invention, has a potential application for the treatment of Gaucher patients.

In another preferred embodiment, the SV40 pseudoviral and/or viral vectors produced by the process of the invention may use as said exogenous nucleic acid sequence, a DNA coding for iRNA (interference RNA). iRNA is a catalytic RNA targeted against specific mRNA, in a process by which double-stranded RNA induces the silencing of homologous endogenous genes [Hammond S M., et al., Nat. Rev. Genet. 2(2):110-119 (2001)]. This iRNA can be used for specific knocking out certain gene products and therefore inhibits and prevents the expression of undesired protein.

In many species, double-stranded RNA can specifically and effectively silence genes. This newly discovered biological phenomenon, called interference RNA (iRNA), has practical implications for functional genomics. As shown by two recent reports, iRNA provides a rapid method to test the function of genes in the nematode Caenorhabditis elegans; most of the genes on C. elegans chromosome I and III have now been tested for iRNA phenotypes. The results validate RNAi as a powerful functional genomics tool for C. elegans, and point the way for similar large-scale studies in other species [Barstead R. et al., Curr Opin Chem Biol. 5(1):63-66 (2001)].

Therefore, the SV40 viruses and pseudoviruses of the invention containing the nucleic acid sequence coding for a specific RNAi, could be used in gene therapy to silence harmful genes, for example oncogenes, or to silence genes of the infecting agent in infections diseases such as hepatitis or HIV.

In an additional embodiment, the SV40 pseudoviral and/or viral vectors produced by the process of the invention may use as said exogenous nucleic acid sequence DNA encoding an exogenous RNA, particularly RNA which encodes a therapeutic protein or peptide product which is not made or contained in a target cell, preferably a mammalian cell, is made or contained in said cell in abnormally low amount, is made or contained in said cell in defective form, and is made or contained in said cell in physiologically abnormal or normal amount, the RNA encoded by said DNA having regulatory elements, including translation signal/s sufficient for the translation of said protein or peptide product in said mammalian cell, operatively linked thereto. The mRNA encoded by the DNA should include mammalian translation signal, for example Kozak sequences. Such constructs will facilitate transient production of proteins, having high specific function in vivo.

Alternatively, the SV40 pseudoviral and/or viral vectors produced by the process of the invention may use as exogenous nucleic acid sequence a DNA encoding an exogenous antisense RNA or ribozyme RNA, or any RNA or is itself a DNA which inhibits or prevents the expression of undesired protein/s or peptide/s in any target cells.

In an additional embodiment the SV40 pseudoviral and/or viral vectors produced by the process of the invention may use as said exogenous nucleic acid sequence a DNA coding for a therapeutic protein or peptide. Such protein or peptide product is, respectively, a therapeutic protein or peptide product which is not made or contained in said cell, or is a therapeutic protein or peptide product which is made or contained in said cell in abnormally low amount, in defective form or is made or contained in said cell in physiologically abnormal or normal amount. The exogenous protein or peptide may be a naturally occurring or recombinant protein or peptide, a chemically modified or peptide, or a synthetic protein and peptide.

In some embodiments the therapeutic proteins may have specific function in the fate of DNA delivery. Thus, the SV40 viral or pseudoviral vectors of the invention will enable the delivery of DNA encoding for a protein which promotes homologous recombination, such as REC A. This technique will enable gene replacement therapy.

The pseudoviral or viral SV40 vectors produced by the process of the invention are suitable for infecting or transfecting any suitable target cell, preferably any mammalian cell. Specific cells are hemopoietic cells, such as bone marrow cell, peripheral blood cells and cord blood cells, or liver cells, epithelial cells, endothelial cells, epidermal cells, spleen cells, fibroblasts, pancreatic cells, muscle cells, tumor cells, cells of the peripheral or central nervous system and germ line cells.

The invention further provides for a therapeutically safe preparation of any one of SV40 viral and pseudoviral vector according to the invention, prepared by the process as defined by the process of the invention.

A further aspect of the present invention relates to the use of the complementation cell line of the invention, for the preparation of a pharmaceutical composition. This composition comprises as an effective ingredient an SV40 viral or pseudoviral vector containing a sequence coding for a therapeutic protein or peptide product according to the invention. The composition according to the present invention is intended for the in vivo and/or ex vivo treatment of a subject in need suffering from any pathological disorder.

By “subject in need” is meant any mammal for which gene therapy is desired, including any mammalian subject such as human, bovine, equine, canine, and feline subjects, preferably, human patients.

The complementation cells of the invention are particularly useful for replication and assembly of T-Ag deleted SV40 vectors which comprising late SV40 genes required for packaging. However, the complementation cell line of the invention may further be used for replication of SV40 vectors comprising only the origin of replication (SV40 ori). In case that assembly of such pseudoviral vectors is desired, late gene products such as genes coding for the capsid proteins (VP1, 2, 3 and agnoprotein) must be provided by introduction of additional helper vector that may be for example co-transfected or co-infected to the complementation cells of the invention. As a non-limiting example for such helper vector is the SLT3 [Oppenheim and Peleg, ibid (1989)].

It is to be appreciated that the complementation cell lines of the present invention, that were prepared by the method described herein and can be use for production of safe SV40 vectors, may be used also as a powerful tool for basic research of different biological and physiological aspects on SV40.

A particular embodiment relates to preparation of pharmaceutical compositions for the treatment of an individual suffering from pathological disorder requiring expression of a product which is normally not made by said individual, or modulation of a product that is made in abnormally low or high amounts or in a defective or abnormal form. Products expressed in defective or abnormal form may be, by non-limiting example, fusion proteins created by translocations and rearrangements occurring in cancer.

The invention also provides a pharmaceutical composition comprising as active ingredient a therapeutically effective amount of one or more defective recombinant SV40 viral or pseudoviral vectors prepared according to a process of the invention and a pharmaceutically acceptable carrier or diluent. The pharmaceutical compositions of the invention can be formulated for any form of administration, preferably topical, oral, parenteral, intranasal, intravenous, intramuscular, subcutaneous, intraocular or transdermal administration.

The pharmaceutical compositions of the invention generally comprise a buffering agent, an agent which adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the intended scope of the invention.

### EXAMPLES

Experimental Procedures

Plasmid Construction

Plasmid pUMTB—This plasmid contains the T-antigen sequences under control of the mouse metallothionein promoter (FIG. 1A), was constructed as follows. A Kpn I-Xba I fragment from plasmid pMT-tss+ [Reiser, W. and Hauser, H. Drug Research 37, 482-485 (1987)] that contains approximately 650 base pairs of the mouse metallothionein promoter was inserted into the Kpn I and Xba I sites in plasmid pUC18. Next, a 260 base pair fragment from plasmid pcDNA3 (Invitrogen), (Xba I to Sph I), that contains the bovine growth hormone polyadenylation sequences, was inserted downstream to the mouse metallothionein promoter into the Xba I and Sph I sites. A PCR fragment that contains the entire SV40 T-antigen coding sequences, from SV40 nucleotide 2687 to SV40 nucleotide 5179, was generated with primers 5′-TAGGCTTCTAGAAAAAGCTTTGCAAAG-3′ also denoted by SEQ ID NO: 1 and 5′-ACAATTGGGCCCATTTTATGTTTCAGGT-3′ denoted by SEQ ID NO:2, and SV40 DNA (Gibco-BRL) as template. Xba I and Apa I restriction sites were incorporated into the forward and the reverse primers, respectively (underlined). The PCR product was digested with Xba I and Apa I and inserted between the mouse metallothionein promoter and the bovine growth hormone polyadenylation element into the corresponding restriction sites. The resulting pUMTB plasmid of the invention carries a short (83 bp) overlap with the SV40 vectors, coordinates 2687-2770, as shown in FIG. 1B.

Plasmid pSLB—is a T-antigen deleted SV40 genome, cloned as a BamH I fragment into pBR322. The T-antigen coding region between the Bcl I site (SV40 nt 2770) and a Hind III site (nt 5171) was replaced by an oligonucleotide carrying Hind II-Xba I-Pst I-Bgl II-Sac I cloning sites.

Plasmid pSLB-EGFP—(FIG. 1C) was constructed by inserting the enhanced green fluorescent protein gene, a Nhe I-Bgl II fragment from pEGFP-C1 (Clontech), into the Xba I-Bgl II sites of pSLB.

Plasmid pSLB-luc (FIG. 1D) was constructed by inserting the luciferase gene, a Hind III-Xba I fragment from pGL3-basic (Promega), into the Hind III-Xba I sites of pSLB.

Cell-Lines

CV-1 (ATCC# CCL-70).

Vero (ATCC# CCL-81).

COS-1 [Gluzman, Y. Cell 23, 175-82 (1981)].

CMT4 [Gerard, R. D. and Gluzman, Y. Molecular and Cellular Biology 5, 3231-40 (1985)].

COT cell-lines—were constructed by co-transfecting pUMTB and pLN into CV-1 and Vero cells, as described herein below.

HuH7 is a human cell-line derived from hepatic cellular carcinoma [Hidekazu Nakabayashi, et al., Cancer Research. 42: 3858-3863 (1982)].

Cells were maintained in Dulbecco's modified Eagle's medium (DMEM) supplemented with 10% fetal calf serum (FCS).

COT Cell-Lines Construction

To construct the COT cell-lines, plasmids PUMTB constructed herein and pLN [Miller, A. D. and Rosman, G. J. Biotechniques 7, 980-990 (1989)] were co-transfected into CV-1 and Vero cells by the calcium-phosphate precipitation method. pLN carries a neoR expression cassette and no SV40 sequences. Before transfection, fragments carrying the expression cassettes were excised from both plasmids and purified following agarose gel electrophoresis. Plasmid pUMTB was digested with Sph I and Kpn I, and pLN with Sac II and Nde I. Forty-eight hours post-transfection the cells were divided into selective medium containing 0.25 μg/ml or 0.4 μg/ml G418 (Sigma). Antibiotic resistant colonies were isolated after 3 weeks.

Vector Preparation

The bacterial sequences were excised off PSLB-EGFP and pSLB-luc by digestion with BamH I and religated at a low DNA concentration (3 microgram/ml) as previously described [Rund, D., et al., Human Gene Therapy 9, 649-57 (1998)]. The ligated vector DNA was transfected into logarithmically growing cultures with the Superfect reagent™ (Qiagen). After transfection COT cells were incubated for 5 days in medium containing 0.1 mM ZnCl₂ and 1 mM CdSO₄ for the induction of T-antigen expression.

In CMT4 cells continuous metal induction leads to cell death. Therefore transfected CMT4 cells were incubated in medium containing heavy metals for 2 days only. The medium was then replaced with fresh medium without heavy metals and incubation continued for 3 additional days.

For COS-1 cells the medium did not contain heavy metals.

Five days post-transfection the vector was harvested by 3 freeze-thaw cycles followed by sonication. Cellular debris were removed by centrifugation at 3200 rpm (2,135×g) for 30 minutes at 4° C. Vector preparations were propagated by repeated cycles of infection and harvesting. Infections were performed by incubating cells with vector stocks (1.2 ml per 75-cm² flask) for 2 hours at 37° C., with gentle rocking every 20 minutes, followed by the addition of fresh medium. Harvest was performed 5 days later, as described above. Vector stocks were stored at −20° C.

It should be noted that in case that the viral vector titers are low, a further step of concentration by purification may be performed according to method well known in the art [Dalyot-Herman, N., et al., Journal of Molecular Biology 256, 69-80 (1996)].

Titration and Luciferse Activity Measurement

Viral vector stocks were assayed as infectious units by the in situ hybridization method on CMT4 monolayers as previously described [Dalyot-Herman, ibid., (1996)]. Briefly, logarithmically growing CMT4 cells in 60 mm-plates were infected with different dilutions of vector preparations in a total volume of 0.4 ml. Following adsorption for 2 hours, T-antigen expression was induced by the addition of heavy metals and vector DNA was allowed to replicate for 2 days. The cell monolayers were then transferred onto nitrocellulose membrane and treated as for colony hybridization [Sambrook, J., et al., Molecular cloning—a laboratory manual. Cold Spring Harbour Laboratory Press, Cold Spring Harbour (1989)]. [³²P]-labeled vector specific DNA was used as hybridization probes.

Luciferase activity was quantified by infecting COS-1 cells with SV/luc vector preparations in a 24-well plate. All infections were performed in triplicate. Forty-eight hours post-infection luciferase activity was analyzed with the Steady-glo™ kit (Promega) and a SPECTRAFluor Plus microplate luminometer (TECAN).

Vector DNA Analysis

Low molecular weight DNA was extracted from infected cells by a modified Hirt procedure [Arad, U. Biotechniques 24, 760-2 (1998)] 3 days post-infection. In this procedure, low molecular weight DNA in Hirt lysates is purified on QIAprep® silica-gel columns (Qiagen). The DNA was analyzed by agarose-gel electrophoresis and Southern Blot Hybridization. A [³²P]-labeled Hind III-fragment of the SV40 early region (nucleotides 4002 to 5171) was used as a probe to detect T-antigen containing recombinants.

Preparation of Viral Vectors for in-Vivo Targeting in Mice

Vector harvesting and concentration included lysing the cells in 0.5% dexoycholate and 1% TirtonX100, pelleting cellular debris by centrifugation and pelleting the virus in the supernatant by ultracentrifugation (80,000 g for 6 hours). The virus pellet was re-suspended in PBS by sonication and residual detergents were removed by batch treatment with Bio-Beads (BioRad).

### Example 1

### Propagation of SV40 Vectors in CMT4 Cells Results in Emergence of Replication-Competent Virus and Loss of Transducing Activity

In CMT4 cells, a 653-bp fragment of the mouse metallothionein promoter has been placed upstream to the T-antigen coding sequence, leaving sequence identity with the vector only downstream to the gene. The present inventors have speculated that eliminating sequence identity at one end of the T-antigen gene would significantly reduce the level of double crossover events and the generation of T-antigen positive recombinants.

SV40 vectors carrying the luc transgene, SV/luc, prepared from plasmid pSLB-luc were propagated by passaging in CMT4 cells, as described in Materials and Methods. Vector stocks were titered for infectious units (IU) as infective centers on CMT4 monolayers [Dalyot-Herman, ibid. (1996)] and in parallel tested for their ability to transduce COS-1 cells. The titer of IU increased with repeated passaging (FIG. 2A) but unexpectedly the luc-transducing activity of the vectors decreased. Analysis of the vector stocks was performed to ascertain whether replication-competent virus had emerged which could explain the discrepancy between the rise in IU titer relative to the loss of transducing activity. Low molecular weight DNA was extracted from COS-1 cells infected with SV/luc preparations. The DNA was analyzed by Southern blotting with a T-antigen specific probe. T-antigen containing vector did in fact emerge in the stocks and the amount of these contaminating vectors increase with repeated passaging (FIG. 2B, lanes 3-5).

To test whether the T-antigen recombinants were replication-competent, CV-1 cells, that do not contain the T-antigen gene, were infected with the vector and low MW DNA was analyzed. Indeed, the T-antigen containing recombinants were capable of independent replication (FIG. 2B, lanes 6-7). The number of replication-competent recombinants increased dramatically from the 4^{th} to 6^{th} passage (compare lanes 6 and 7 in FIG. 2B) presumably due to the selective advantage of propagation of wt SV40 under these conditions. These findings prompted us to construct a packaging cell-line that would preclude homologous recombination.

### Example 2

### Construction of a T-Antigen Expression Cassette Containing Minimal Homology to SV40 Vector Sequences

SV40 vectors contain sequences that include the overlapping early and late polyadenylation signals, up to the Bcl I site at SV40 coordinate 2770, including a 77 bp overlap with the T-antigen coding region (FIG. 1B). The early polyadenylation signal is usually utilized for transgene expression, and the late, that is in closer proximity to the T-antigen coding sequence, for expression of the capsid proteins required for vector production. Hence, development of a packaging cell-line that supplies the T-antigen in trans, without any sequence identity to the vector, requires modifications of the vector itself. Plasmid PUMTB (FIG. 1A), in which T-antigen transcription is driven by the mouse metallothionein promoter and utilizes the bovine growth hormone polyadenylation signal, have been constructed. This strategy limits the identity between the packaging cell-line and the vector sequences from over 2 kb (as in COS- and CMT4 cells) to only 83 base pairs at the 3′ end of the T-antigen coding sequence. This is below the stretch of identity required for efficient homologous recombination in mammalian cells, which is estimated to be between 200 to 300 base pairs [Liskay, R., et al., Genetics 115, 161-167 (1987)].

Following cloning, the ability of pUMTB to support replication of an ori containing plasmid in response to heavy-metal (Zn⁺⁺, Cd⁺⁺) induction, have been confirmed by the inventors. CV-1 cells were co-transfected with pUMTB and pSO3cat, which contains SV40 ori [Oppenheim, A., et al., Proc. Nat. Acad. Sci. USA. 83, 6925-9 (1986)]. Cells were then incubated with and without metal induction for 48 hours. Low molecular weight DNA was subsequently extracted, digested with DpnI to remove unreplicated input DNA and analyzed by Southern blotting. In the presence of heavy metals, pUMTB was able to induce the replication of pSO3cat (not shown), indicating expression of functional T-antigen that can drive SV40 ori dependent replication.

### Example 3

### Isolation of Cell-Lines that Carry an Inducible T-Antigen

The bacterial sequences were excised off PUMTB. The linearized expression cassette was transfected into CV-1 and Vero cells, together with a linear neoR expression cassette which does not contain any SV40 sequences, derived from pLN [Miller, A. D. and Rosman, G. J. Biotechniques 7, 980-990 (1989)]. Forty-eight hours post transfection cells were replaced in selective medium containing G418 for 3 weeks, until antibiotic resistant colonies could be isolated. Individual colonies were expanded and screened for T-antigen expression by RNA analysis. Five of the CV-1 clones and 3 of the VERO clones expressed elevated T-antigen RNA in response to metal induction.

These cell-lines were then evaluated for their ability to package SV40 vectors. SV/GFP vector DNA was excised with BamH I from pSLB-GFP (FIG. 1C), then circularized by self-ligation and transfected into expanded clones. The SV/GFP viral vectors were harvested by freeze-thaw and sonication and vector production was assayed by infecting COS-1 cells. GFP expression was estimated by fluorescent microscopy. The results showed that the Vero derivatives were poor in vector production. Three of the CV-1 derivatives, COT2, COT4 and COT18, were effective in packaging SV/GFP vectors and were selected for further studies.

In order to increase the vector titers, the inventors proceeded to optimize conditions by varying the incubation time from transfection to vector harvest, the duration periods of heavy metal induction, medium volume and FCS concentration. The optimal conditions are those presented in Experimental procedures.

### Example 4

### Characterization of the Packaging Cell Line

Transducing Activity is Lost After Repeated Passaging

Initial studies with these packaging cell-lines using a number of vectors carrying glucocerebrosidase [Reiner, O., et al., DNA 6, 101-108 (1987) ], LNGFR [Schilz, A., et al., Blood 92, 3163-3171 (1998)] and lmrA [Van Veen, H., et al., Nature 391, 291-295 (1998)], suggested some irreproducibility in the preparation of stocks titered as infective centers. Moreover, the inventors have noted that stocks with a titer of 10⁶ to 10⁷ IU/ml and even higher sometimes gave poor results when transgene expression was measured. To further investigate this issue, GFP was cloned into the vector. The titration assay of the invention, scores infective particles that enter and replicate in CMT4 cells, and GFP is a most suitable reporter gene to measure transducing particles. Vectors prepared on COT2 were analyzed in parallel to vectors prepared in COS-1 cells (FIG. 3A). As expected, the IU titer of SV/GFP vectors prepared in COT2 cells increased with repeated passaging from 5×10⁴ IU/ml after the first harvest to 2×10⁶ IU/ml in the 3^{rd}-passage. However, the titer of GFP transducing units decreased in the second passage and was undetectable in the 3^{rd}-passage. SV/GFP vectors propagated in COS-1 cells showed a moderate decrease in the titer of both IU and transducing units. Similar results were obtained when SV/GFP vectors were propagated from a different plasmid, pSLE-GFP, in which the vector DNA is inserted as an EcoR I fragment into a pUC backbone (not shown). These results were reproduced three times. The inventors speculated that this might be due to GFP toxicity, which has been reported by several investigators [Liu, H., et al., Biochemical and Biophysical Research Communications 260, 712-717 (1999)].

Transducing activity of SV/GFP vectors could not be increased by repeated passaging in neither COS-1 nor COT2 cell-lines. This could be attributed to the potential toxicity of GFP [Liu, H. et al.,(1999)] or alternatively, to factors inherent to the primary base sequence of the GFP gene. The primary nucleotide sequence of the transgene may affect the rate of vector rearrangements. Experiments in our laboratory with other transgenes imply that the nature of the transgene influences the level of defective vectors that accumulate. This is possibly due the predilection of certain transgene sequences to interfere with DNA replication and/or encapsidation.

Interestingly, it appears that different cell-lines have characteristic rate patterns at which defective vector appear. This may be due to variability in the expression of proteins that function in non-homologous recombination. It has been suggested that the SV40 T-antigen promotes DNA recombination [Cheng, R., et al., Experimental Cell Research 234, 300-312 (1997)] by activating human RAD51 expression and also through binding and sequestering the p53 protein [Xia, S., et al., Molecular and Cellular Biology 17, 7151-7158 (1997)]. Therefore, while high levels of T-antigen are required for vector DNA-replication, they may also enhance the formation of defective vectors. The level of expression of T-antigen in each COT cell-line is probably dictated by the context of the integration site of the expression cassette.

Further experiments were conducted with the luc reporter gene. The three COT cell-lines, COT2, COT4 and COT18 were tested for vector production. The IU titer of SV/luc vector preparations increased with additional passages, reaching 4-7×10⁶ IU/ml in the 5^{th}-passage in all cell-lines assayed (FIG. 3B). To measure the transducing activity of these SV/luc stocks, COS-1 cells were infected with the same vector preparations and luc activity was assayed by luminometry 2 days post infection. The transducing activity of the SV/luc stocks did not correlate with their IU titer (FIGS. 3B and 3C). The results showed that luc-transducing activity of vectors prepared in the COS-1 and COT18 cell-lines increased in the initial passages, but in subsequent passaging (between passage 3 and 5) these vectors began to lose their transducing activity (FIG. 3C). In vector prepared on COT2 and COT4, luc-transducing activity logarithmically decreased with every passage. By the 4^{th}-passage, luc activity was barely above background. The results suggested extensive recombination in the COT cell-lines.

It is generally thought that SV40 recombination is enhanced by a high multiplicity of infection (MOI). The inventors have noted that significant accumulation of defective vectors was repeatedly observed at an MOI lower than 0.1 in various experiments.

Vectors Acquire Deletions During Repeated Passaging

The possibility that generation of non-transducing defective vectors is due to non-homologous recombination was next investigated. Vectors propagated in COT2 cells were analyzed by infection using a SV/luc stock that was propagated for 5 passages. Low molecular weight DNA was subsequently extracted. The DNA was linearized and analyzed by agarose gel electrophoresis in parallel to the input vector DNA used for the initial transfection. Two bands were observed in 5^{th} -passage SV/luc DNA (FIG. 4, lane 2), both representing DNA of lower MW DNA than the input vector (FIG. 4, lane 1), demonstrating the accumulation of deletions during repeated passaging. Similar results were obtained for a SV/GFP vector that was propagated for 3 passages. Here, a single band with lower MW than the input SV/GFP vector was observed (FIG. 4, lanes 4 and 5).

Plaque-forming SV40 is not Produced in Vectors Passaged on COT Cells

A major concern for gene therapy is the emergence of replication-competent, T-antigen bearing viral particles, which may propagate in the host. The possibility that the recombination events described above could lead to re-acquisition by the vector of the T-antigen sequences present in the packaging cells, was next inquired. High passage (5^{th}) SV/luc vector stocks prepared on COS-1 and the various COT cell-lines were screened for plaque-forming units (PFU) by the standard plaque assay on CV-1PD cells. Table 1 shows that replication-competent virus was not detected in four different stocks prepared on COT cell-lines. In additional experiments no replication-competent virus was detected in five additional stocks prepared on COT18. One of two stocks prepared on COS-1 cells was contaminated by wild-type SV40 at a ratio of ˜1 PFU per 10⁴ IU.

TABLE 1
IU titer and PFU titer of SV/luc stocks prepared
on various cell-lines

| | IU Titer (/ml) | PFU (/ml)^{a} |
|---|---|---|
| COT2 #1 | 1 × 10⁷ | 0 |
| COT2 #2 | 4.4 × 10⁷ | 0 |
| COT4 | 3.8 × 10⁷ | 0 |
| COT18 | 7 × 10⁷ | 0 |
| COS-1 #1 | 1.5 × 10⁷ | 0 |
| COS-1 #2 | 5 × 10⁷ | 6.3 × 10³ |

^{a}The sensitivity of detection was 3 PFU/ml.

The Rate of Appearance of Defective Vectors is Cell-Line Specific

The discrepancy between IU and transducing activities provides a measure for the magnitude of defective-vector accumulation in a given stock. The results shown in FIG. 5 are representative of several independent experiments in which the inventors consistently found that luc-transducing activity was lost more rapidly in COT2 and COT4 than in COT18 and in COS-1. The following experiment was performed in order to obtain additional evidence of the cell-specific nature of this process. A 2^{nd}-passage SV/luc stock prepared in COT2 cells was divided and further passaged in parallel in both COT2 and COS-1 cells. Remarkably, luc-transducing activity of the vector preparations that were transferred from COT2 to COS-1 cells started to increase, while the vector that continued to be passaged on COT2 cells continued to accumulate defective vectors (FIG. 5). Transfection and passaging exclusively in COS-1 cells, in a parallel experiment, showed a continuous increase in luc-transducing activity. These results suggested that cell-specific host factors influence that rate at which defective vectors accumulate.

### Example 5

### Transduction of a Human Hepatic Cell-Line

The inventors have next compared the transduction efficiency of human hepatic cells by viral vectors prepared using the COT18 cells of the invention and vectors prepared using the well known COS-1 cells. Therefore, SV/luc vectors were prepared on COT18 and on COS-1 and luc transducing activity of each vector stock was measured by infecting COS-1 cells grown in 24-well plates in triplicate and measuring luc activity 2 days post-infection with the Steady-glo™ kit (Promega) and a SPECTRAFluor Plus microplate luminometer (TECAN).

HuH7, which is a human cell-line derived from hepatic cellular carcinoma used as a model for gene therapy and was utilized by the inventors for evaluating the transduction efficiency of the vectors prepared by the packaging cell of the invention. Sub-confluent cells grown in 24 well plates were infected in triplicates, 100 microliter each, with SV/luc vector stocks which were prepared on COS-1 and on COT18 cells. Two days later the cells were analyzed for luciferase activity. The results of the triplicates were averaged and normalized to the titer of the vector stocks (measured as luc transducing activity on COS cells, as detailed above). The results (FIG. 6) show higher transduction efficiency of the vector prepared on COT18 (244 relative units) compared to the vector prepared on COS-1 (95 relative units), demonstrating that SV40 vectors prepared on COT18 are very efficient in transduction of human liver cells.

### Example 6

### In-Vivo Targeting of Liver Tissue by Viral Vectors Prepared in COT18 Cells

The therapeutic potential of viral vectors prepared by the packaging cell line of the invention was next evaluated by examining the in-vivo tissue targeting capacity of these viral vectors. In-vivo tissue targeting to mouse liver was performed by injecting 10⁷ transducing units of SV/luc vector (prepared on COT18 cells), through the tail vain of a BALB/C mouse. Following three passages in COT18 cells, the vector was harvested and concentrated as described in Experimental procedures. In order to target the liver, the vector was injected into the tail vein using the high volume (1 ml) high pressure (3-5 sec) technique. Luciferase expression was analyzed 1 day post-injection using a cooled charge-coupled device (CCCD) camera. Light emission was monitored five minutes after animals were injected IP with Beetle luciferin. As demonstrated by FIG. 7, a significant expression of luciferase is shown in the live mouse, indicating a correct in-vivo tissue targeting and transgene expression of the viral vector prepared in COT18 cells.

1. A complementation SV40 packaging cell line for in-trans complementation of SV40 T-antigen (T-Ag), which cell line eliminates the generation of viable virus carrying the T-Ag gene, by reducing homologous recombination in packaging process of an SV40 vector, wherein said cell line is transformed with at least one expression cassette having minimal sequence identity to SV40 sequences comprised within said SV40 viral vector, said expression cassette comprising:
a. nucleic acid encoding SV40 T-Ag;
b. a heterologous promoter;
c. heterologous termination signal; and optionally
d. additional operably linked control elements and/or selectable markers.

2. The packaging cell line according to claim 1, optionally further transformed with an additional expression cassette comprising a gene coding for a selectable marker.

3. The packaging cell line according to claim 1, wherein the SV40 sequences comprised within the cells and the SV40 sequences comprised within said SV40 viral vector share a sequence identity of between 0 and about 300 nucleotides.

4. The packaging cell line according to claim 3, wherein the SV40 sequences comprised within the cells and the SV40 viral vector share 83 nucleotides corresponding to nucleotide 2770 to 2687 of the SV40 genome as denoted by GenBank Accession Number J20400.

5. The packaging cell line according to claim 1, wherein the T-Ag is expressed under the control of a heterologous inducible promoter.

6. The packaging cell line according to claim 5, wherein said inducible promoter is the mouse inducible metalloprotein promoter.

7. The packaging cell line according to claim 1, wherein said heterologous termination signal is a polyadenylation sequence.

8. The packaging cell line according to claim 7, wherein said heterologous polyadenylation sequence is a bovine growth hormone polyadenylation signal.

9. The packaging cell line according to claim 1, wherein said DNA encoding SV40 T-Ag is a DNA fragment corresponding to nucleotides 5179 to 2687 of the SV40 genome as denoted by GenBank Accession Number J20400.

10. The packaging cell line according to claim 9, wherein said expression cassette is comprised within a plasmid designated pUMTB and comprises a DNA fragment encoding SV40 T-Ag corresponding to nucleotides 5179 to 2687 of the SV40 genome, expressed under the control of the inducible mouse metalloprotein promoter and a bovine growth hormone polyadenylation signal.

11. The packaging cell line according to claim 2, wherein said additional expression cassette is comprised within a plasmid designated pLN, which plasmid comprises a sequence coding for neomycine resistance.

12. The packaging cell line according to claim 1, transformed with the plasmids pUMTB and pLN.

13. The packaging cell line according to claim 1, wherein said complementation line is derived from any one of primate SV40 permissive and semi-permissive primary cells and cell lines.

14. The packaging cell line according to claim 13, wherein said primate SV40 permissive and semi-permissive cell line is derived from cells lines selected from the group consisting of Vero, CV-1, Hela, and BSC1 cells.

15. The packaging cell line according to claim 14, wherein said complementation line is derived from CV-1 cell line.

16. The packaging cell line according to claim 15, wherein said cells are selected from the group consisting of COT2, COT4 and COT18.

17. The packaging cell line according to claim 16, wherein said cell line is the COT18 cell line deposited under DSM Accession No. ACC2525 or any cell line derived therefrom.

18. An expression cassette for the production of a complementation SV40 packaging cell line, having minimal sequence identity to SV40 sequences comprised within an SV40 viral vector, said expression cassette comprising:
a. nucleic acid sequence coding for SV40 T-Ag;
b. a heterologous promoter;
c. a heterologous termination signal; and
d. optionally additional operably linked control elements and/or selectable markers.

19. The expression cassette according to claim 18, wherein said heterologous promoter is an inducible promoter.

20. An expression cassette according to claim 19, comprising a DNA fragment coding for SV40 T-Ag corresponding to nucleotides 5179 to 2687 of the SV40 genome as denoted by GenBank Accession Number J20400, the inducible mouse metalloprotein promoter and a bovine growth hormone polyadenylation signal.

21. The expression cassette according to claim 20, wherein said cassette is comprised within a plasmid designated pUMTB.

22. A method for the production of a complementation SV40 packaging cell line for in-trans complementation of viral T-antigen (T-Ag), which method comprises the steps of:
a. providing primate SV40 permissive or semi-permissive cells;
b. transforming said cells with at least one expression cassette as defined by claim 18 and optionally further transforming said cells with an additional expression cassette comprising a gene coding for a selectable marker; and
c. selecting for transformed cells expressing the expression cassette as defined by claim 18.

23. A process for the production of a preparation comprising recombinant T-Ag-deleted SV40 viral and/or pseudoviral vector and devoid of T-Ag containing recombinant SV40 viral and/or pseudoviral vector, comprising the steps of:
a. providing a complementation packaging cell line according to claim 1;
b. infecting or transfecting said cell line with any one of SV40 viral or pseudoviral vector;
c. culturing the infected cells under suitable conditions for permitting the production of said SV40 viral or pseudoviral vectors; and
d. harvesting the viruses.

24. The process according to claim 23, wherein said complementation packaging cell line is the COT18 cell line deposited under DSM Accession No. ACC2525 or any cell line derived therefrom.

25. The process according to claim 23, wherein said recombinant T-Ag-deleted SV40 viral and/or pseudoviral vector comprises an exogenous nucleic acid sequence encoding an exogenous protein or peptide product, or encoding a therapeutic RNA, or encoding an antisense RNA, ribozyme RNA, or encoding an iRNA (interference RNA) or is itself a therapeutic product which inhibits or prevents the expression of undesired protein/s in any target cell and optionally additional operably linked control elements

26. A T-Ag-deleted SV40 viral and/or pseudoviral vector produced by the process of claim 23.

27. The SV40 viral vector according to claim 26, wherein said vector comprises an exogenous nucleic acid sequence encoding an exogenous protein or peptide product, or encoding a therapeutic RNA, or encoding an antisense RNA, ribozyme RNA, or encoding an iRNA (interference RNA) or is itself a therapeutic product which inhibits or prevents the expression of undesired protein/s in any target cell and optionally additional operably linked control elements.

28. The SV40 viral vector according to claim 28, wherein said therapeutic protein or peptide product is at least one of a therapeutic protein or peptide which is not expressed, is expressed in abnormally low amount, is expressed in defective form or is expressed in physiologically abnormal amount, in a target cell.

29. The SV40 viral vector according to claim 28, wherein said therapeutic protein or peptide product is at least one of an enzyme, a receptor, a structural protein, a membrane protein, a regulatory protein and a hormone.

30. A therapeutically safe preparation of any one of SV40 viral and pseudoviral vector according to claim 26.

31. A pharmaceutical composition comprising as an effective ingredient any one of an SV40 viral and pseudoviral vector according to claim 26.

32. Use of a complementation cell line according to claim 1 in the preparation of a pharmaceutical composition.

33. Use of a T-Ag-deleted SV40 viral and/or pseudoviral vector according to claim 26 in the preparation of a pharmaceutical composite.
